(19) United States
(12) Patent Application Publication
(10) Pub. No.: US 2003/0036725 A1
(43) Pub. Date: Feb. 20, 2003

(54) Reconstitution and injection system

(76) Inventors: Gilad Lavi, Rishon Lezion (IL); Gjl Yigal, Gan-Yavne (IL); Jonathan Bar-Or, Pardess Hana (IL)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 09/957,179

(22) Filed: Sep. 20, 2001

Related U.S. Application Data
(60) Provisional application No. 60/234,118, filed on Sep. 21, 2000.

(51) Int. Cl. A61M 37/00; A61M 31/00

(52) U.S. Cl. 604/91; 604/236

(57) ABSTRACT

This reconstitution and injection system delivers a drug in solution under pressure, and injects powdered or lyophilized drugs that require reconstitution, rehydration or dilution. In one embodiment, the system includes a syringe having a barrel, a plunger, a handle, a pump and a channel communicating the barrel with an injection needle. The pump pushes a liquid from the barrel through the injection needle via the channel upon fluid communication between the barrel and the injection needle for delivery of the liquid. In another embodiment, the system includes a first port receiving a syringe, a second port receiving a drug vial, a channel providing communication between the first and second ports, and a controller permitting or inhibiting fluid communication between the first and second ports. In yet another embodiment, the system includes an actuator and a housing having a channel providing fluid communication between a reservoir and the injection needle.

Previously, various devices have been developed for the delivery of medications into and through the skin of living organisms. These devices include syringes in which a liquid drug solution is delivered through the skin of a user from a syringe chamber by the manual movement of the syringe plunger to move the drug solution from the chamber through the syringe needle inserted under the skin.

The liquid can be a mixture of the drug (e.g., powdered, lyophilized, concentrated liquid) and a diluent (e.g., dextrox solution, saline solution, water), since certain injectable substances (e.g., glycogen, used to dissolve blood clots) do not maintain their chemical and physical stability when mixed with a diluent and thus cannot be stored for a substantial period of time. Therefore, powdered, concentrated or lyophilized substances (e.g., drugs or compounds) are presently used for injection of materials that would otherwise be unstable. Lyophilization, for example, is the rapid freezing of a material at a very low temperature followed by rapid dehydration by sublimation in a high vacuum. The resulting lyophilized compound is typically stored in a glass vial or cartridge which is closed by a cap, such as a rubber stopper or septum.

Prior to administration of the injectable substances, it is necessary to reconstitute the concentrated or solid material (e.g., lyophilized compound). Reconstitution, for example, is accomplished by mixing the concentrated or solid compound with a suitable diluent or liquid. Reconstitution typically involves the use of a syringe with a needle to withdraw the diluent from a separate vial and inject it into the vial containing the compound. The compound is then thoroughly mixed, typically by swirling the vial by hand, and a separate syringe with a needle withdraws the desired amount to be injected into the patient.

Because two separate containers are used, the person reconstituting the compound must be certain to mix the correct amounts of the compound and diluent to achieve proper concentration of the mixture. Generally, when a syringe is used to mix the diluent and drug, the desired volume of diluent to drug ratio is difficult to obtain. Thus, precious concentration levels of administered drugs may be compromised, as it is generally not possible to go back and fix mistakes of overdose or drain air bubbles. Moreover, with air being used to push liquids through the system, the possibility of air bubbles is increased. It would be beneficial to provide a drug delivery device that allows a user (e.g., medical personnel, patient, person delivering the mixture) to easily correct injection problems prior to delivery (e.g., concentration, overdose, air bubbles).

In addition, some drug applications require the implementation of several vials during a single application. For example, during an application of fertility hormone, the reconstitution process may include seven vials having different concentricity levels of the same or different drugs. It would be beneficial if these applications could be provided by one drug delivery device.

Furthermore, sometimes when injecting a drug, it is difficult to determine the end of delivery without eye contact with the applicator. Therefore, it would be beneficial if an applicator would provide a clear indication of the end of the drug delivery, so that no eye contact would be required.

Because of the increased use of powdered and concentrated compounds, and lyophilized drugs, for example, it is desirable to provide both professional and nonprofessional personnel with a reconstitution and injection system. It is desirable to have a simple, reliable system that facilitates safe preparation and delivery of an accurate dosage of a reconstituted compound. In addition, it is desirable to provide a system that reconstitutes a lyophilized drug while maintaining sterility throughout the process. Also, it is desirable to provide improvements in the subcutaneous delivery of medication generally, which provide for a safe, effective administration by the user. Moreover, it is desirable to provide a system that reduces needlephobia.

### SUMMARY OF THE INVENTION

The present invention relates to systems and methods for delivering a drug compound to a user. In a preferred embodiment, the system includes a housing including a first recess communicating with a drug cartridge and a second recess communicating with a plunger assembly. The housing also includes a spring loaded actuator for moving a delivery needle from the system housing into a user for injection.

A standard syringe can be modified for use as the plunger assembly within this system by adding a spring around the rod of a syringe plunger between an attachment at a distal end of the plunger and a piston at a proximal end thereof. The attachment (e.g., handle or clip) has a larger diameter than the distal end of the plunger and the syringe body. The syringe may be used as a prefilled syringe or it may be empty and added with diluent or a drug solution prior to use.

The reconstitution and injection system further includes a first pathway from the drug cartridge or vial to the syringe and a second pathway from the syringe to a chamber in liquid communication with the delivery needle. The delivery needle has an opening (e.g., notch) thereon which provides liquid communication between the hollow interior of the delivery needle and the chamber.

In a preferred embodiment, an injection device comprises a syringe having a barrel, a plunger, a handle and a pump. The barrel has a drug reservoir therein. The reservoir is arranged to have a liquid therein. The plunger is slidingly located within the barrel and coupled to the plunger. The pump is located between the plunger and the handle. The injection device also includes a first channel arranged for selectively providing fluid communication between the drug reservoir and a hollow injection needle. The pump is arranged to drive the liquid from the drug reservoir through the injection needle via the first channel upon the fluid communication between the drug reservoir and the injection needle to deliver the liquid to an injection site.

In another preferred embodiment, a syringe comprises a barrel having a drug reservoir arranged to hold a liquid therein, a plunger slidingly engaged within the barrel, a handle coupled to the plunger, and a pump located between the plunger and the handle. The drug reservoir and the injection needle are arranged to be selectively placed in fluid communication with each other, and the pump is arranged for driving the liquid from the drug reservoir through an injection needle upon the fluid communication between the drug reservoir and the injection needle for delivery of the liquid to an injection site.

In another preferred embodiment, a mixing device is coupled to a syringe having a drug reservoir therein. The mixing device includes a first port arranged for receiving the syringe, the syringe having a barrel coupled to or integral with the first port, the barrel having a drug reservoir therein, the drug reservoir being in fluid communication with the first port. The mixing device also includes a second port arranged for receiving a vial, the vial having an interior in fluid communication with the second port, a first channel arranged for selectively communicating the first port with the second port, and a pathway controller in fluid communication with the first channel and between the first and second ports. The pathway controller is arranged for permitting the fluid communication between the first and second ports when the controller is in a first position and for inhibiting the fluid communication between the first and second ports when the controller is in a second position.

In another preferred embodiment, an injection device comprises an actuator and a housing. The actuator has a hub and a hollow injection needle, the hub holding the injection needle, the injection needle having a tip at its distal end and an opening proximal to the tip. The housing is coupled to the actuator, the housing including a channel arranged to selectively provide fluid communication between a liquid reservoir holding a liquid and the injection needle. The hub is arranged for moving the injection needle from a first position where the opening in the injection needle is not in fluid communication with the channel to a second position where the opening in the needle is in fluid communication with the channel to enable expulsion of a liquid from the liquid reservoir.

The invention also includes a method for delivering liquid or reconstituted powdered drugs to a user by inserting and locking a syringe (e.g., prefilled) into a syringe recess, inserting the drug cartridge into a drug cartridge recess. In this preferred embodiment, a spike or needle located at the bottom of the drug cartridge recess pierces a rubber stopper of the drug cartridge to open a passageway from the drug cartridge to the prefilled syringe.

According to this method of the invention, a plunger slidingly engaged within the handle is depressed into the syringe housing, whereupon the diluent is moved from the syringe into the drug cartridge (e.g., vial). After the diluent solution is reconstituted with the drug in the vial, the handle and rod of the syringe are pulled back which causes the reconstituted drug solution to move from the drug vial into the syringe. The position of a piston within the syringe can be adjusted to the appropriate level of solution for injection.

The first pathway from the vial to the syringe is closed, to lock the drug solution in the syringe. The handle is depressed until it engages and locks with the syringe housing, thereby squeezing the spring against the piston and placing the drug solution under pressure. The spring loaded actuator is depressed to move the delivery needle outside the injection system housing, which places the delivery needle opening in position to provide liquid communication via the second pathway to the syringe. This communication releases the fluid lock of the drug solution. Upon this release, the plunger spring extends and pushed the piston, thus forcing the pressurized drug solution through the delivery needle for injection into the patient.

In a method of ejecting a fluid from a barrel of a syringe having a handle, a barrel, a plunger and a hollow needle, the barrel having the fluid therein, the preferred method comprises securing the handle of the syringe to the barrel to bias the plunger against the fluid in the barrel placing the fluid under pressure, and actuating a movement of the needle to place the hollow interior of the injection needle in fluid communication with the fluid whereupon the plunger automatically pushes the fluid through the injection needle.

In a method of ejecting a liquid from a barrel of a syringe, the preferred method comprises depressing the handle of the plunger to cause the plunger to slide within the barrel to couple the handle to the barrel to place the liquid under pressure, and actuating a movement of the injection needle within the syringe for insertion into an injection site. The movement of the injection needle establishes fluid communication between an interior of the injection needle and the liquid which releases the pressure of the liquid in the barrel and enables the plunger to push the liquid through the injection needle.

In another method of ejecting a liquid from a barrel of a syringe, the preferred method comprises applying a bias against the plunger in communication with the liquid to place the liquid under pressure, actuating a movement of the injection needle within the syringe for insertion into an injection site. The movement of the injection needle establishes fluid communication between the hollow interior of the injection needle and the liquid. The fluid communication releases the pressure of the liquid in the barrel and enables the compression pump to push the plunger within the barrel and force the liquid through the injection needle.

In a method of mixing a drug in a vial with a liquid from a reservoir to form a drug compound, and ejecting the drug compound from a reservoir in an injecting device, the injecting device having an injection needle, a plunger located within the reservoir, and a control valve therebetween, the preferred method comprises adjusting the control valve to establish fluid communication between the liquid in the reservoir and an interior of the drug vial via the control valve, the interior of the drug vial containing the drug, transferring the liquid from the reservoir to the interior of the drug vial, the liquid mixing with the drug to form the drug compound, transferring the drug compound to the reservoir, adjusting the control valve to terminate fluid communication between the reservoir and the interior of the drug vial, biasing the plunger against the drug compound in the reservoir to place the drug compound under pressure, and actuating a movement of the injection needle, the movement of the injection needle establishing fluid communication between the interior of the injection needle and the drug compound. The communication releases the pressure of the drug compound in the reservoir and enabling the plunger to push the drug compound through the injection needle.

Further scope of applicability of the present invention will become apparent from the detailed description given hereinafter. However, it should be understood that the detailed description and specific examples, while indicating preferred embodiments of the invention, are given by way of illustration only, since the invention will become apparent to those skilled in the art from this detailed description.

### BRIEF DESCRIPTION OF THE DRAWINGS

The invention will be described in conjunction with the following drawings in which like-referenced numerals designate like elements, and wherein:

FIG. 1 is a longitudinal sectional view showing a system constructed in accordance with a preferred embodiment of the invention for reconstituting a drug in a vial for ultimate delivery into a patient;

FIG. 2 is a transverse sectional view of the reconstitution and injection system taken along line 2-2 of FIG. 1;

FIG. 3 is a view similar to that of FIG. 1, but showing the system in the state wherein its syringe's plunger is depressed to carry a diluent into the vial;

FIG. 4 is a view similar to that of FIG. 1 but showing the syringe's plunger in a retracted state and a solution pathway button in a closed position to prevent the diluent from entering into the vial;

FIG. 5 is a transverse sectional view taken along line 5-5 of FIG. 4;

FIG. 6 is a view similar to FIG. 4, but showing the handle or cap of the syringe's plunger releasably secured to the syringe's housing;

FIG. 7 is a longitudinal view partially in section of the system of FIG. 6 taken in a direction 180 degrees from that of FIG. 6 to show the delivery needle of the system held in a retracted position by an actuator of the system;

FIG. 8 is a view similar to FIG. 6, but showing the delivery needle of the system in its extended position;

FIG. 9 is a view similar to FIG. 7, showing an actuator in an operative state whereupon the delivery needle is placed in its extended position;

FIG. 10 is a view like that of FIG. 8 after delivery of the drug compound;

FIG. 11 is a view like that of FIG. 9 after delivery of the drug compound;

FIG. 12 is a view like that of FIG. 9, but showing the system in its locked out state wherein the delivery needle is locked in a retracted position to prevent reuse;

FIG. 13 is a view like that of FIG. 10, but showing the system in its locked position like that of FIG. 12;

FIG. 14 is an isometric view of a reconstitution and injection system in accordance with another preferred embodiment of the invention;

FIG. 15 is an exploded isometric view of the system of FIG. 14;

FIG. 16 is a longitudinal sectional view of the system taken along line 16-16 of FIG. 14;

FIG. 17a is a view similar to that of FIG. 16, but showing the system in the state wherein its plunger is depressed to transfer air into the vial;

FIG. 17b is an enlarged partial view of the system shown in FIG. 17a;

FIG. 18 is a view similar to that of FIG. 16, but showing the syringe's plunger in a retracted state to draw a drug solution into the syringe;

FIG. 19 is a view similar to that of FIG. 16, but showing a different vial communicating with the system;

FIG. 20 is a view similar to that of FIG. 16, but showing the system in the state wherein its plunger is depressed to push the solution into the vial;

FIG. 21 is a view similar to that of FIG. 16, but showing the plunger in a retracted state to draw the reconstituted solution into the syringe;

FIG. 22 is an isometric view of the system similar to that of FIG. 14, but showing the system at a different state;

FIG. 23 is a partial longitudinal sectional view of the system of FIG. 22;

FIG. 24 is a transverse sectional view of the system showing the pathway lever in a forward position;

FIG. 25a is a longitudinal sectional view of the system taken along line 25-25 of FIG. 22;

FIG. 25b is a partial longitudinal sectional view of the system of FIG. 25a;

FIG. 26a is a transverse sectional view of the system taken along line 26-26 of FIG. 22;

FIG. 26b is a partial transverse sectional view of the system of FIG. 26a;

FIG. 27 is a longitudinal sectional view similar to that of FIG. 25a, but showing the delivery needle of the system in its extended position;

FIG. 28 is a transverse sectional view similar to FIG. 26a, but showing the delivery needle of the system in its extended position;

FIG. 29 is a longitudinal sectional view similar to FIG. 27 after delivery of the drug compound and showing the system in its locked-out state, wherein the delivery needle is locked in a retracted position to prevent reuse; and

FIG. 30 is a transverse view like that of FIG. 28, but showing the system in its locked position like that of FIG. 29.

### DETAILED DESCRIPTION OF THE INVENTION

The present invention is directed to reconstitution and injection systems and methods for delivering a drug in solution under pressure, and to the injection of powdered or lyophilized drugs that require reconstitution, rehydration or dilution. The system includes a reconstitution subsystem, a pressurization subsystem, a transfer subsystem and an injector subsystem each of which will be described hereinafter. The reconstitution subsystem includes a drug vial containing powdered, lyophilized, dehydrated or concentrated drugs that receive a diluent for mixing with the contained drug. The pressurization subsystem includes a syringe that places a liquid drug solution under pressure until an opening is provided at its distal end to push the pressurized solution out of the syringe. The transfer subsystem includes passageways, in communication with the reconstitution, pressurization and injection subsystem, that control the ingress/egress of fluids between the aforementioned subsystems. The injection subsystem includes an actuator that places a needle in communication with the transfer system and extends the needle out of the system for receiving and injecting the drug into a patient. The drug is held under pressure by a biasing force, and is automatically released through the needle upon extension of the needle into an injection site.

It should be pointed out at this juncture that the embodiments of the system shown in the Figures include all four subsystems. However, different embodiments of the present invention may use only one or any combination of the subsystems, depending on the requirements of different applications. For example, a preferred embodiment can inject a liquid drug and not require reconstitution. Therefore, the system for such an application need not include a reconstitution subsystem. Alternatively, the reconstitution system may be used to reconstitute or lyophilize a solid drug into solution for subsequent delivery by a standard syringe.

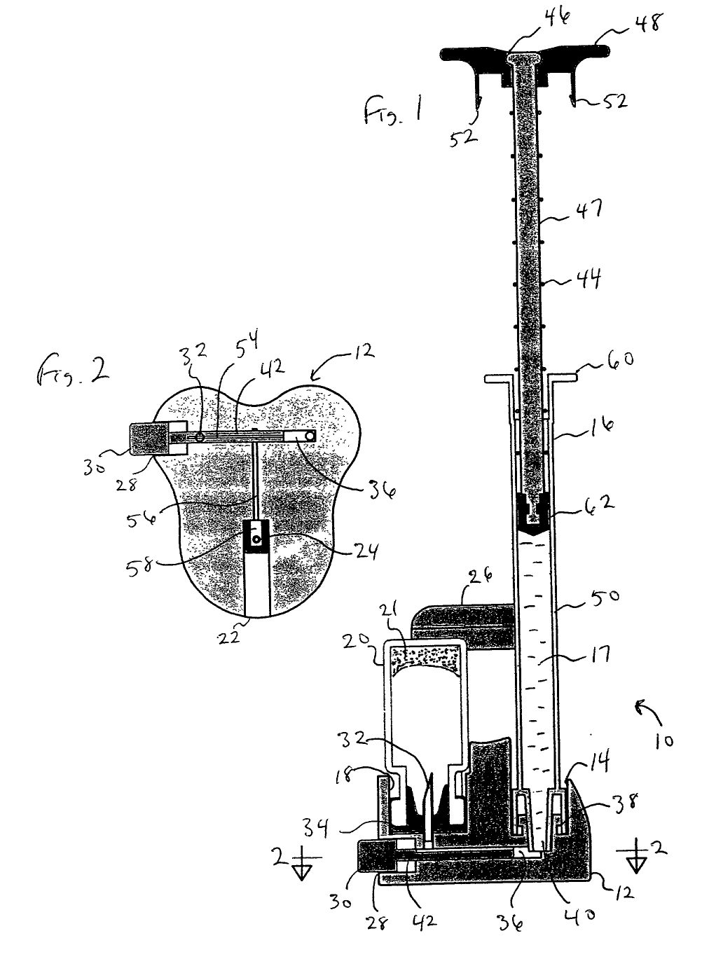

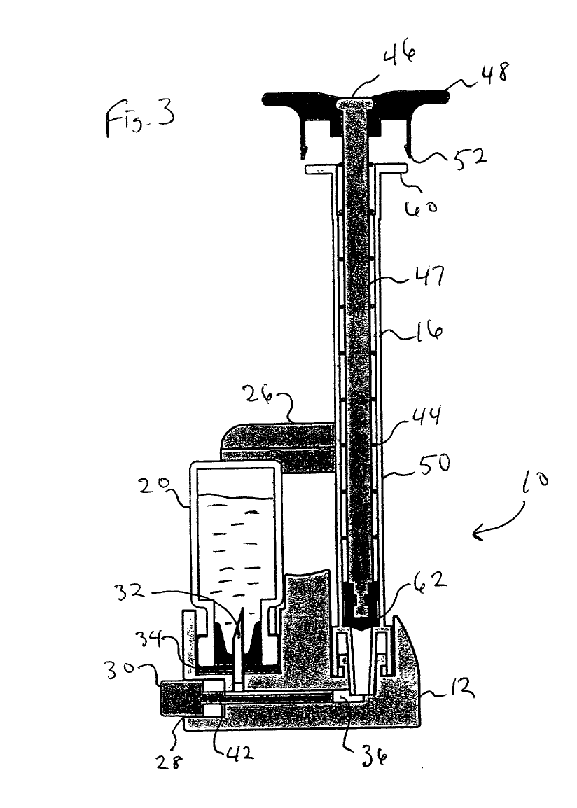

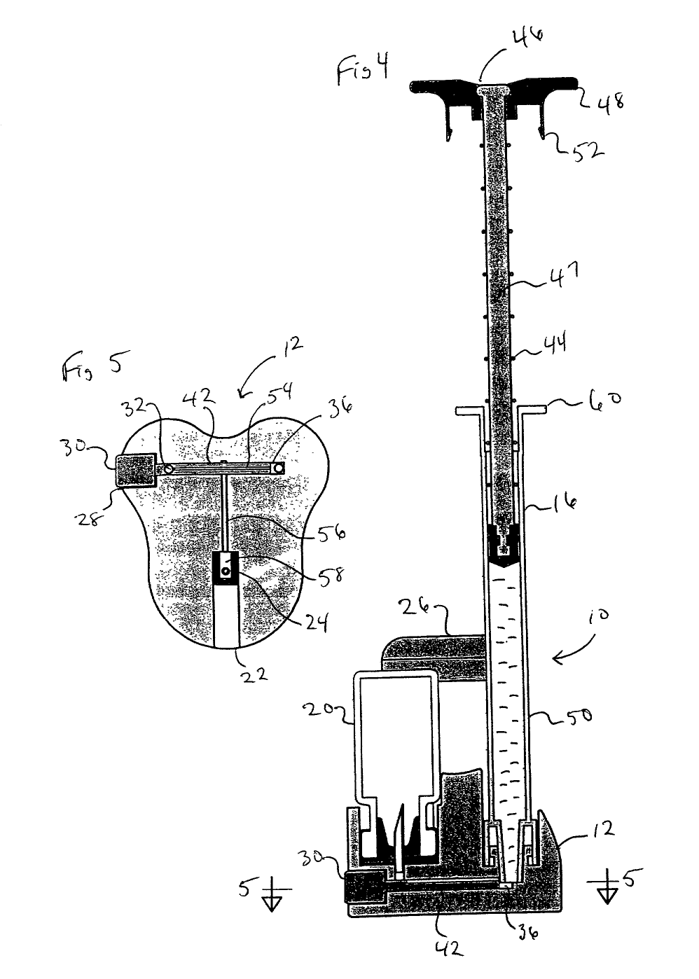

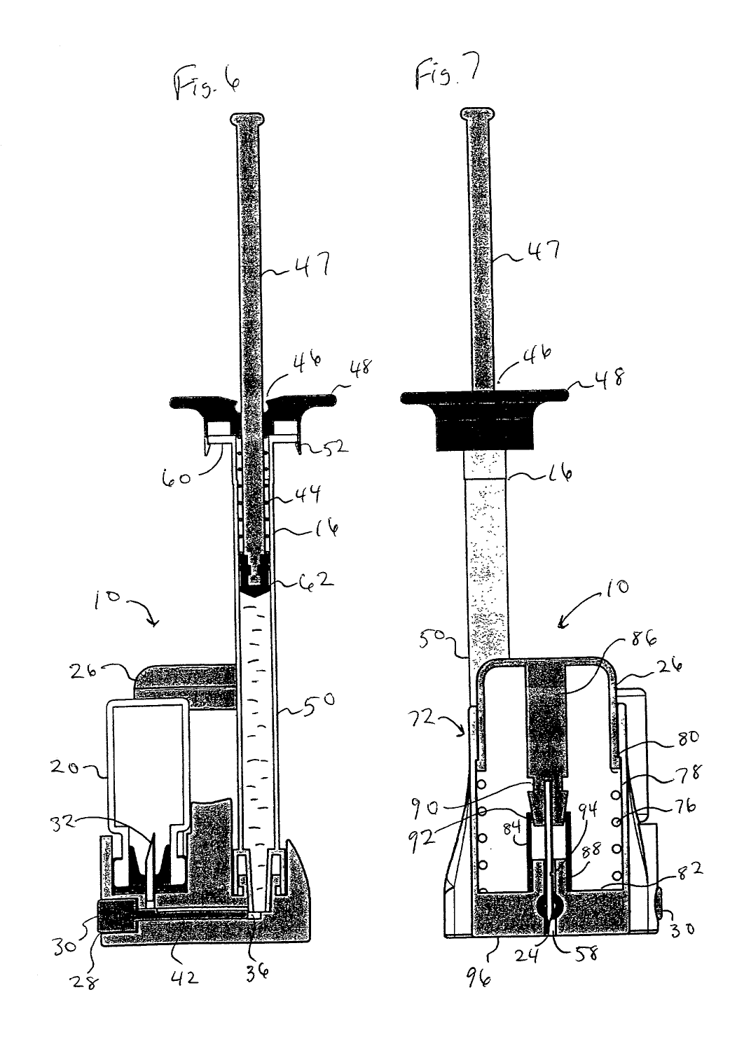

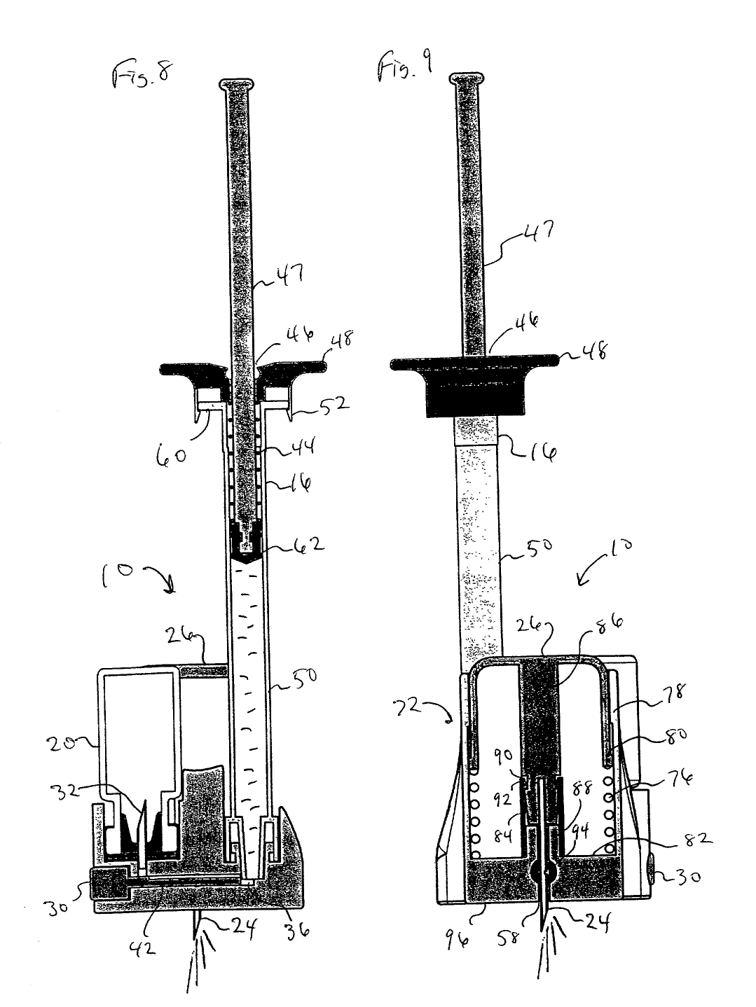

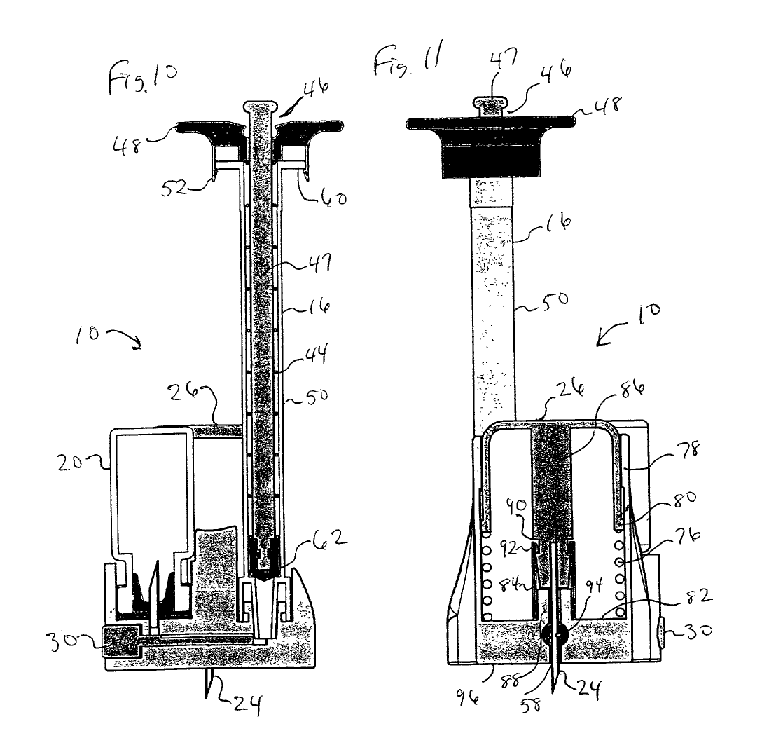

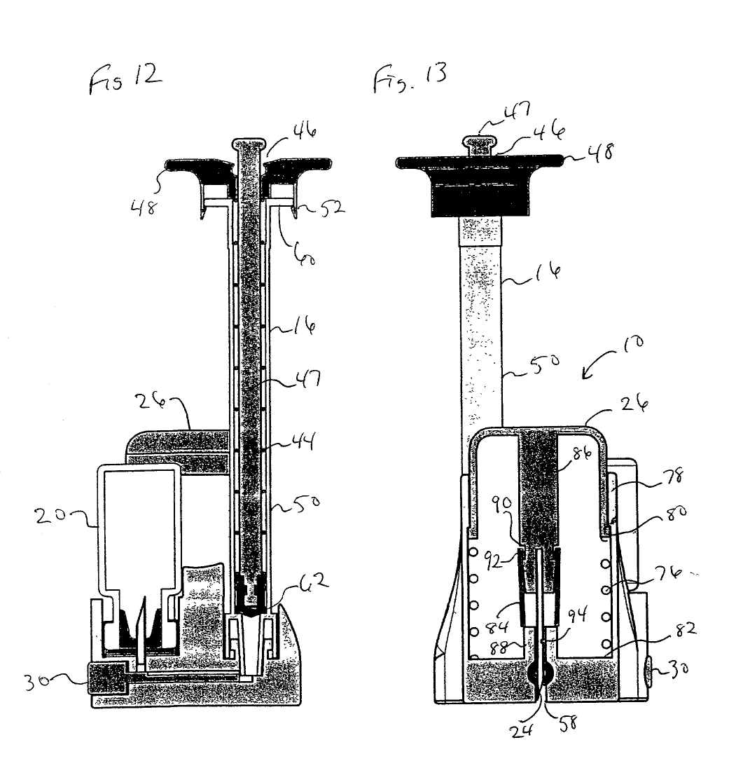

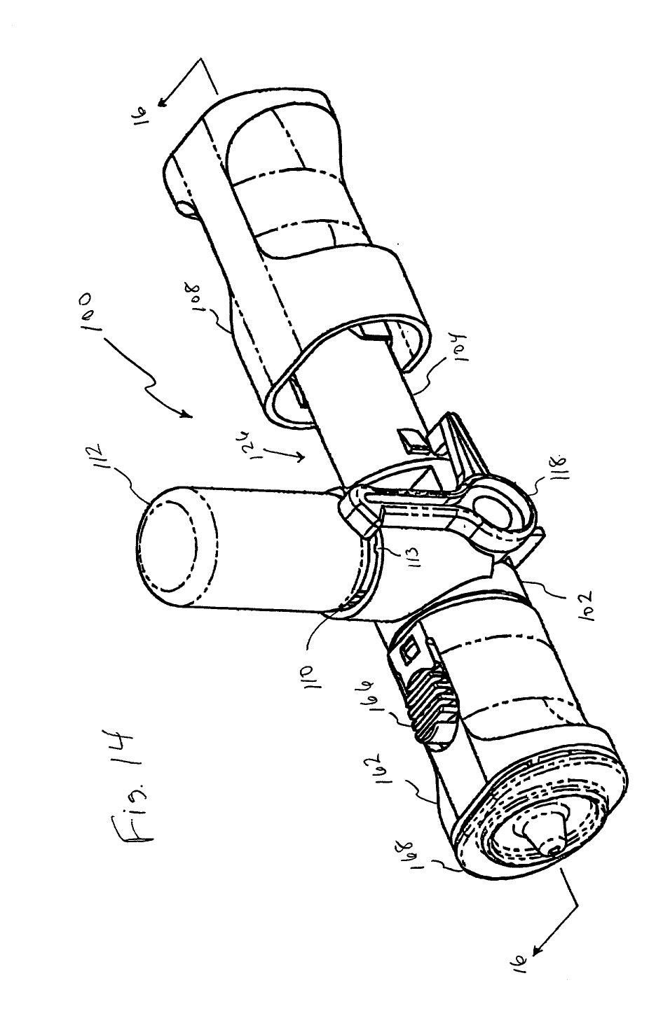

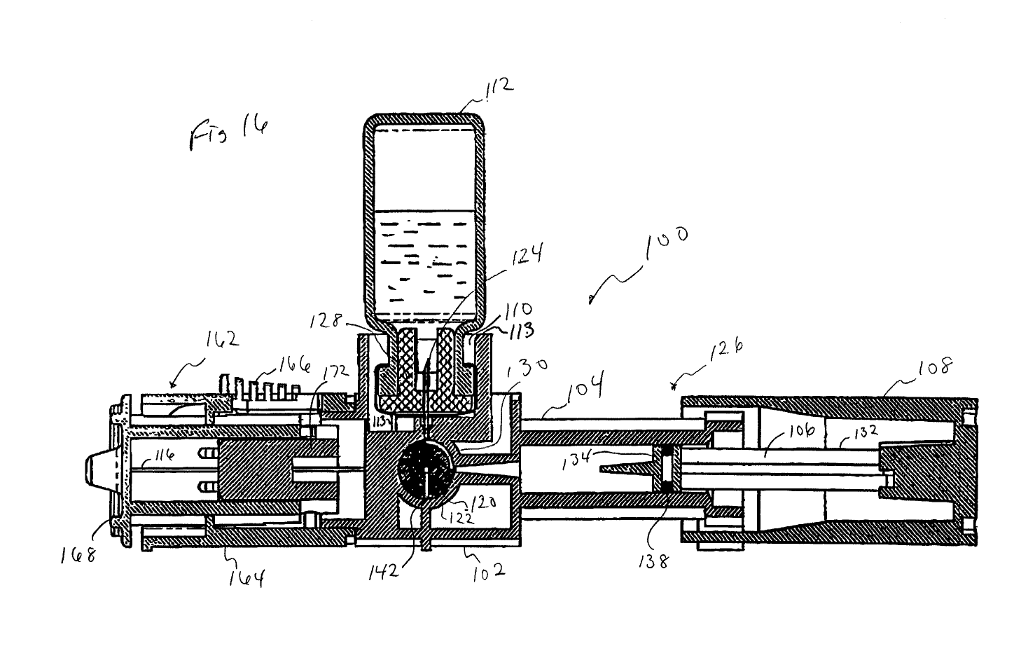

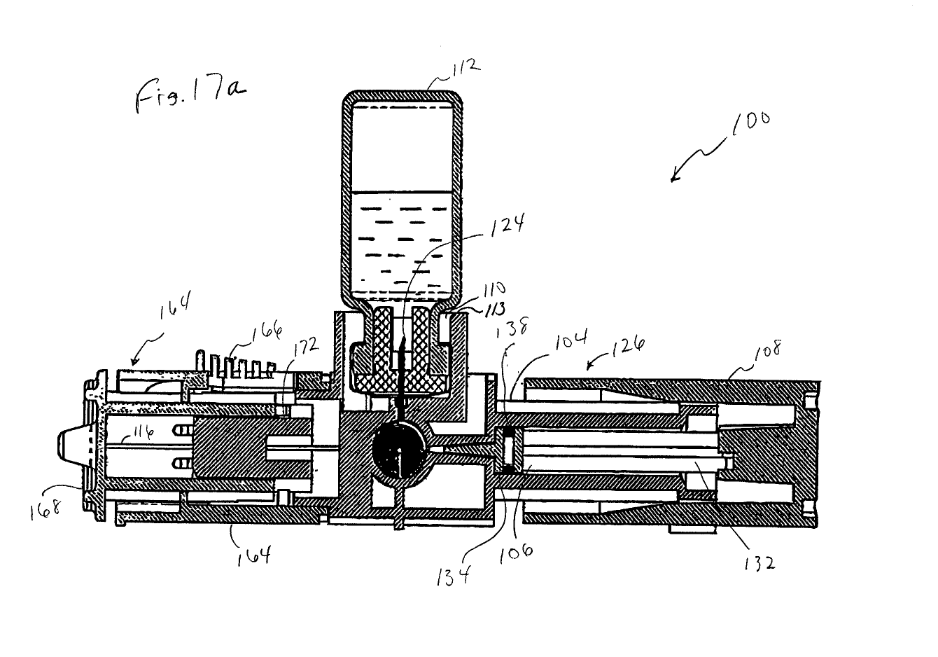

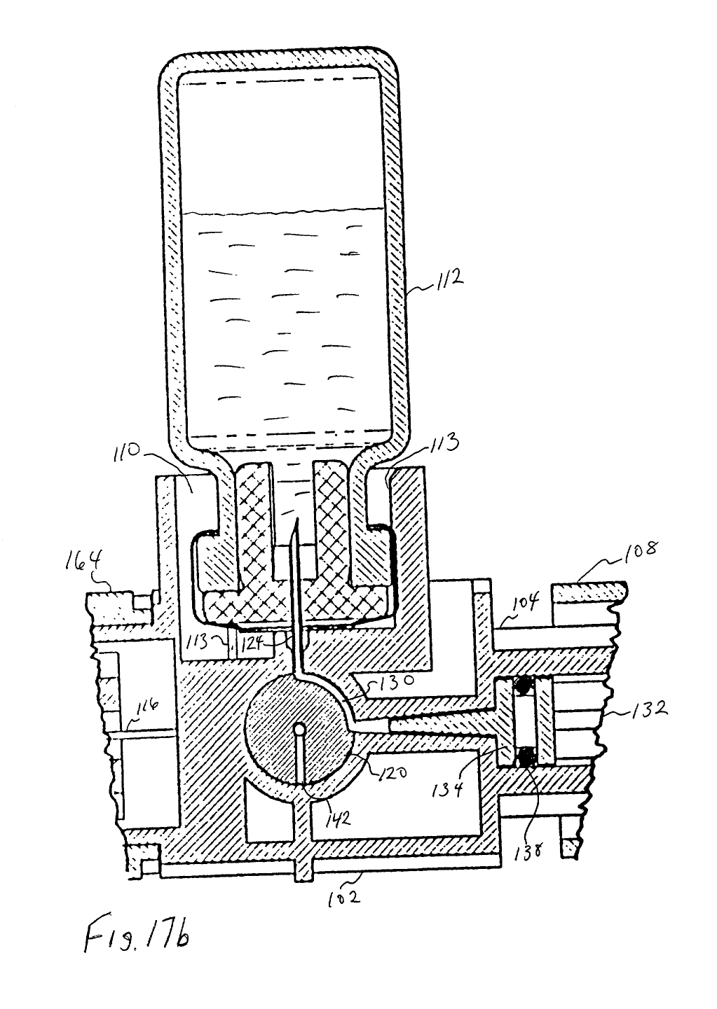

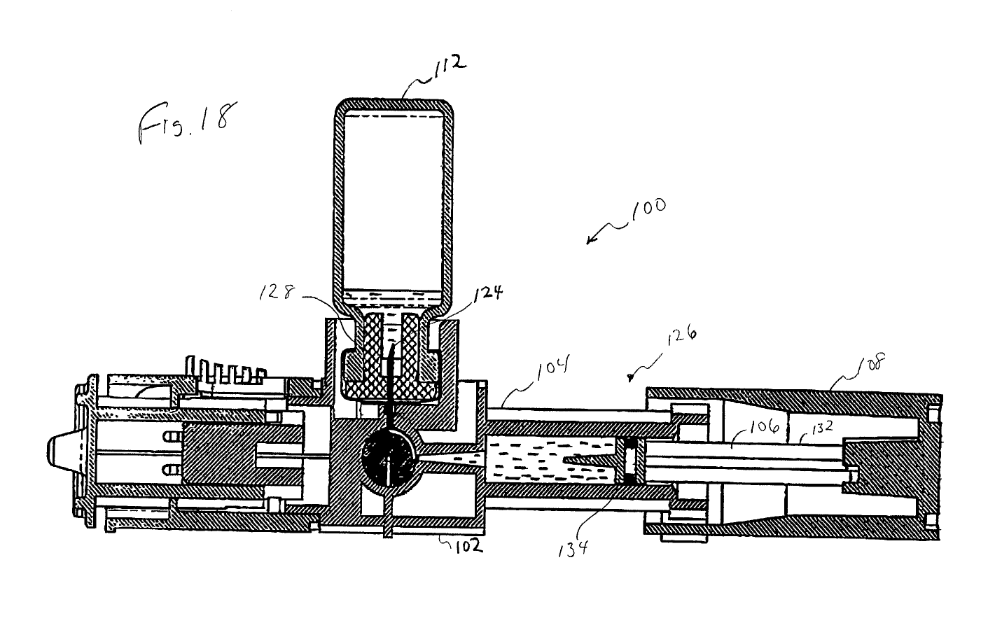

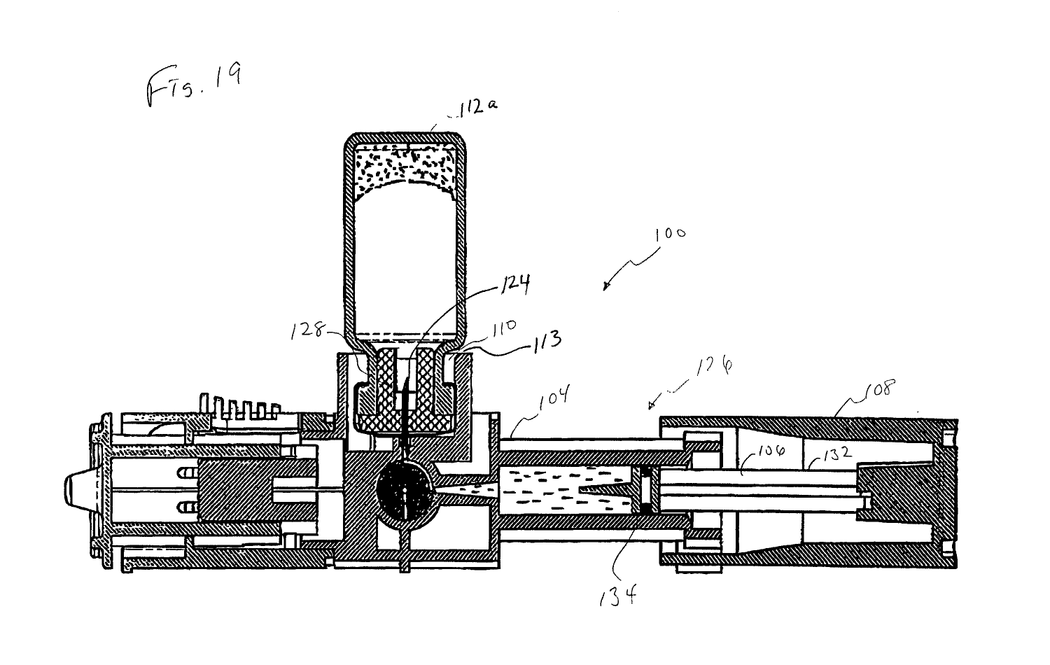

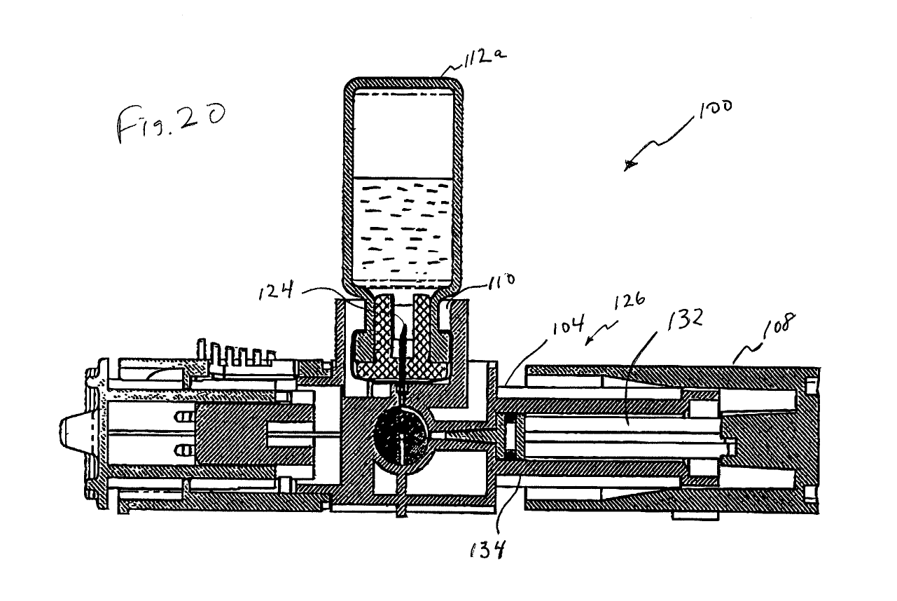

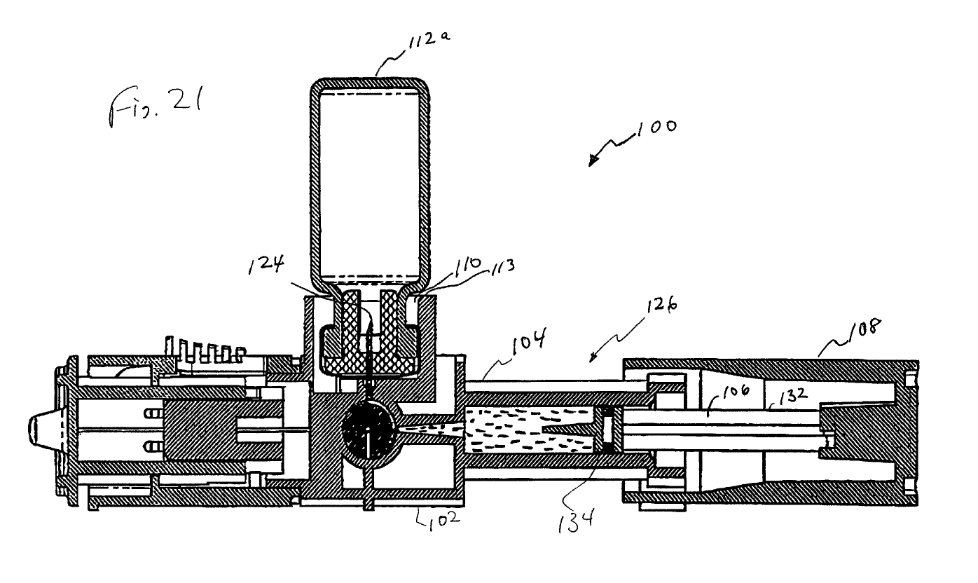

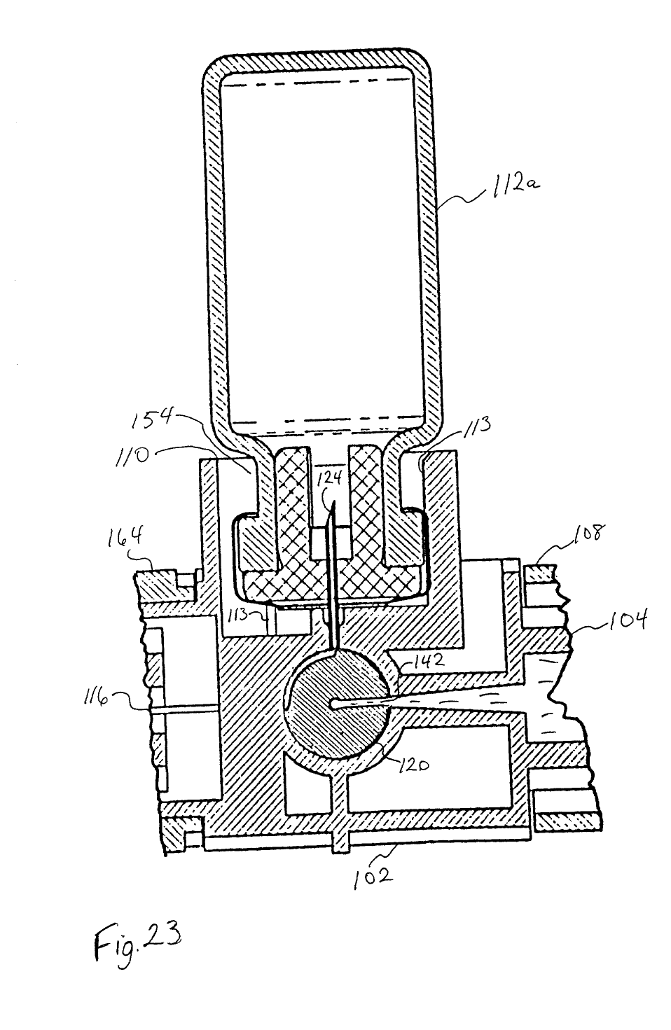

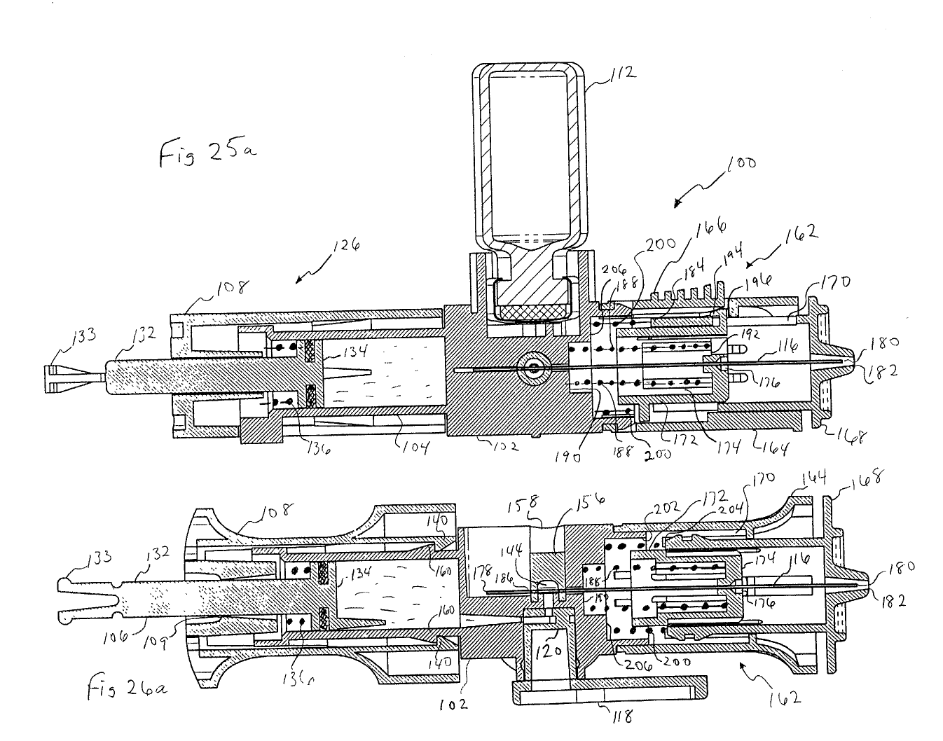

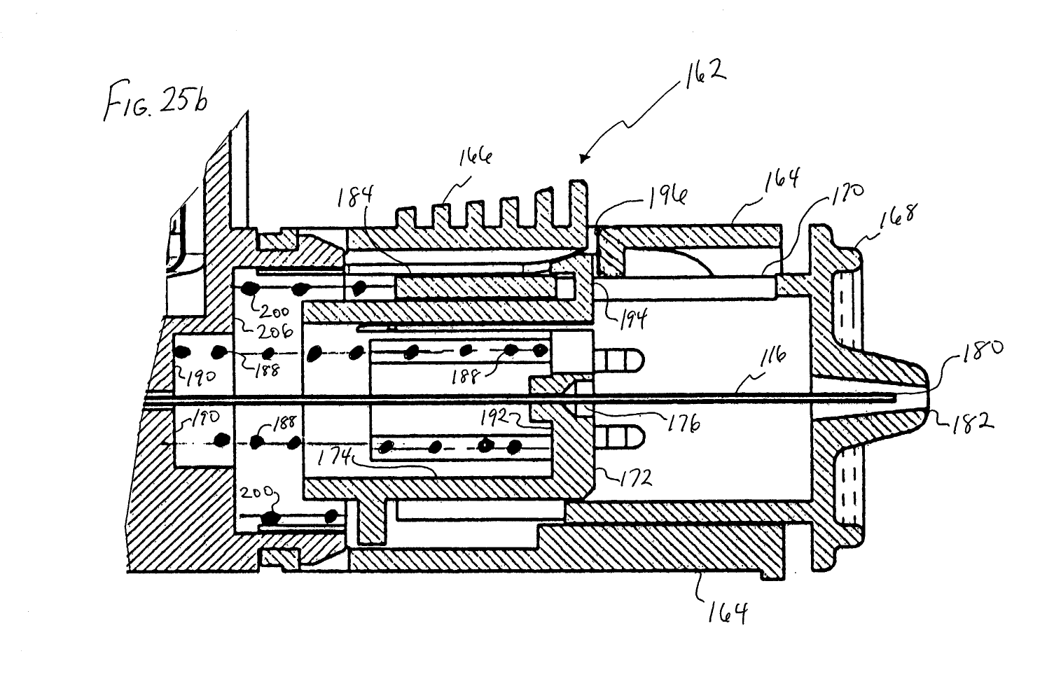

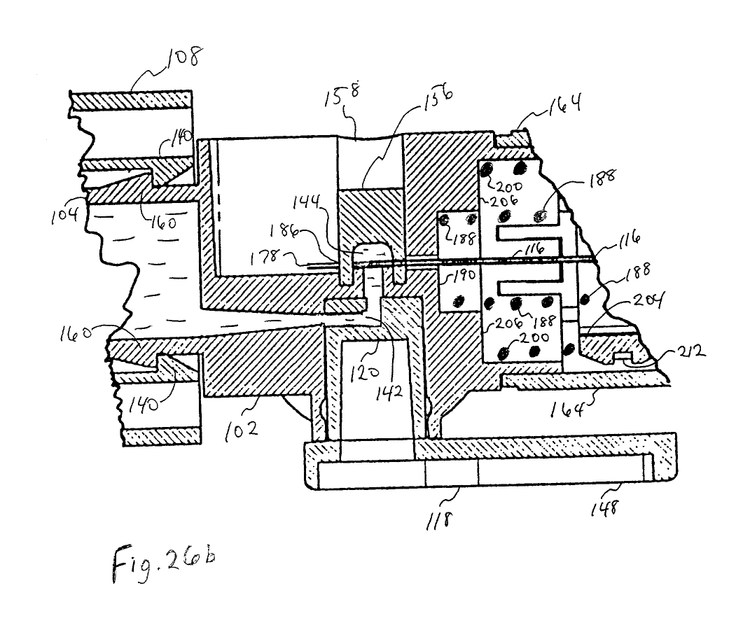

Referring to FIGS. 1 and 2, there is shown at 10 a reconstitution and injection system constructed in accordance with one preferred embodiment of this invention. The system includes a housing 12 formed of any suitable material, e.g., plastic or metal, having a first recessed port or opening 14 for receiving a syringe (conventional or otherwise) 16, and a second recessed port or opening 18 for receiving a drug cartridge or vial (conventional or otherwise) 20. The housing 12 also includes a third recessed port or opening 22 (FIG. 2) for receiving a delivery needle 24 (FIG. 11). The delivery needle 24 serves as a means for delivering the reconstituted drug to the patient. The delivery needle 24 preferably has a penetration length of about 7 mm. However, the penetration length of the delivery needle 24 is not limited to a length of 7 mm since it is understood that the delivery needle may be any length and thickness (e.g., 26 gage) sufficient to penetrate the skin and deliver the drug compound.

A spring loaded actuator 26 is coupled to the opening 22 for moving the delivery needle 24 from the housing 12 into the drug receiver (e.g., patient, or intravenous administration set) for injection, as shown in FIG. 11 (to be described later). The housing 12 also includes a pathway or control button 30 having a stem 42 and located in a bore 28 for controlling the flow of a liquid or drug solution within housing 12 (as will be described later). A needle 32 is located within the housing in the second recessed port 18.

In the embodiment of FIG. 1, the housing 12 has a somewhat cloverleaf-like transverse cross section, although the shape of the housing is only limited by the requirements in use thereof For example, if an additional port or opening were required to receive a second vial, the shape of the housing 12 would be altered accordingly to provide an area for the additional port or opening. In this embodiment, the syringe 16 is prefilled with a diluent 17 and the vial 20 contains a lyophilized drug or compound concentrate 21. Alternatively, the syringe 16 may be empty and the diluent 17 added prior to use. In either case, as shown in FIG. 2, the vial 20 is pressed into the recessed port 18 until the needle 32 penetrates the rubber stopper 34 of the vial that seals the vial 20, such that the needle 32 extends into the interior of the vial 20. The needle 32 is hollow and acts as a passageway through which the diluent 17 from the syringe 16 may flow into the vial 20 when the syringe's plunger 46 (to be described later) is depressed. To that end, as shown in FIG. 3, the needle 32 communicates with a first channel or passageway 36 in the housing that extends to the recessed port 14 receiving the distal end of the syringe 16. Locking tabs (e.g., luer) 38 are provided at the proximal end 40 of the syringe 16 to interlock the syringe within the recessed port 14 and prevent removal therefrom. Upon insertion of the vial 20 into the recessed port 18, the needle 32 pierces the rubber stopper 34 of the vial 20, thus opening the first channel 36 from the vial 20 to the syringe 16. Pressing the syringe's plunger, like shown in FIG. 3, causes the diluent 17 to flow out of the syringe into channel 36 and through needle 30 into the vial 20.

The channel 36 can be closed to prevent access from the vial 20 to the syringe 16 by pressing the control button 30 into the housing 12 to the position shown in FIG. 4 such that the stem 42 blocks and closes the outlet of the channel 36 serving as the pathway to the needle 32. In use it is preferable that the control button 30 is initially positioned so that its stem 42 precludes communication between the vial 20 and syringe 16 until both the vial 20 and syringe 16 are inserted into the housing 12 such that the diluent 17 in the syringe 16 or the drug compound 21 in the vial 20 are not spilled through the first channel 36 into the recessed openings 14 or 18. Once both containers (e.g., syringe 16 and vial 20) are locked into place with the housing 12, the interior of the syringe 16 is in fluid communication with the interior of the vial 20.

The syringe 16, which may be a standard or conventional syringe, includes the heretofore mentioned plunger 46. The plunger is slidingly located within a tubular section (e.g., barrel 50) of the syringe 16 that contains the diluent 17. When using a standard syringe 16, a helical compression spring 44 is provided about the plunger 46 between the plunger's handle 48 and a piston 62 located on the distal end of the plunger 46. The piston 62 is formed of an elastomeric material and its outer diameter is just slightly greater than the inner diameter of the barrel 50 to form a sliding seal therewith so that no diluent can gain egress through the interface of the piston 62 and the barrel 50. The piston thus makes sliding frictional engagement with the inner wall of the barrel 50 for pushing or pulling a solution out of or into the barrel 50. The plunger's handle 48 is in the form of a larger diameter cap. A pair of clips 52 extend downward from the handle or cap 48 for connecting to a flange 60 at the proximal end of the barrel 50, as will be described below.

As mentioned earlier and as shown in FIG. 2, the housing 12 includes the channel 36 that provides a pathway between the drug vial 20 and the syringe 16. That channel intersects with a second channel 56. The second channel 56 provides a pathway from the syringe 16 to the injection needle 24, which is slidingly engaged within a chamber 58, as will be described in greater detail below. In this preferred embodiment of the system 10, the housing 12 provides the communication between the syringe 16, drug vial 20, and injection needle 24.

FIGS. 3-13 generally illustrate the various steps for reconstitution and injection of the drug compound in accordance with the method of use of the system 10. To that end as shown in FIG. 3, the syringe's plunger 46 is depressed into the barrel 50 until the diluent 17 has been moved from the syringe 16 into the vial 20. The system 10 shows the plunger 46 fully depressed into the barrel 50 of the syringe 16 to push the fluid (e.g., diluent) into the vial 20 for mixing with the lyophilized or powdered drug compound. The system 10 is swirled by, for example, a user, to further insure complete reconstitution of the drug/diluent solution.

FIG. 4 illustrates the relative positions of the plunger 46 in the syringe 16 and the button stem 42 in the housing 12. As shown in FIG. 4, the plunger 46 and handle 48 are pulled back which causes the reconstituted solution to move from the drug vial 20 into the syringe 16. In a preferred embodiment, the syringe 16 and/or housing 12 includes a series of visual indications thereon to enable an accurate measurement of the level of solution drawn into the barrel 50 of the syringe 16. Therefore, a user can adjust the position of the piston 62 within the barrel 50 to the appropriate level of solution for injection.

Once the plunger 46 moves the desired amount of reconstituted solution from the drug vial 20 into the syringe 16, the pathway button 30 is pushed further into the housing 12 and closes the liquid communication between the drug cartridge 20 and the syringe 16. As shown in FIGS. 4 and 5, as the pathway button 30 is pushed into the housing 12, the stem 42 blocks the passage from the first opening 14 to the second opening 18, thereby blocking off liquid communication between the drug vial 20 and the syringe 16. At this point, the drug solution within the syringe 16 has nowhere to move.

The handle 48 of the plunger 46 is then depressed onto the outer flange 60, as shown in FIGS. 6 and 7. This depression causes the spring 44 along rod 47 to compress between the piston 62 and the cap or handle 48, placing the drug solution in the barrel 50 under pressure. Clips 52 on the lower side of the handle 48 engage and lock about the outer flange 60 of the syringe 16 to maintain the compression of the spring 44 until the drug solution in the barrel 50 of the syringe 16 is released.

FIG. 7 is a longitudinal view taken opposite the view of the system 10 shown in FIG. 6 to show the drug injection subsystem 72. The drug injection subsystem 72 includes a spring-biased pushing member or actuator 26. The actuator 26 includes a cup shaped upper section having a centrally located upper arm 86. The upper arm mounts the injection needle 24 and holds it in a first position (to be described later). The actuator 26 also includes a cup shaped lower section fixedly secured to the housing 12. The lower section has a bottom wall or floor 82 from which a centrally located lower arm 88 projects upward. A bore 58 extends through the bottom wall and through the lower arm 88. The bore 58 intersects the channel 56. When the injection needle is held in the first position within the bore 58, it blocks the channel 56 so that no drug solution can escape via the syringe 16. A helical compression injection spring 76 is located within the interior of the upper and lower sections of the actuator 26 immediately adjacent the inner surface of the sidewall 78 between the lower end of the upper section 80 and floor 82 of the lower section. A collar 84 extends about the lower arm 88.

When the handle 48 is locked against the flange 60, placing the drug solution under pressure, as shown in FIGS. 6 and 7, the system 10 is ready for injection. In one aspect of this preferred embodiment, if the System 10 is self-adhering, the user or patient then peals a paper lining off of an adhesive layer 96 on the bottom of the housing 12 and applies the bottom of the housing 12 to the appropriate injection site.

As shown in FIGS. 8 and 9, the upper section of the actuator 26 is pressed down to extend the delivery needle 24 out of the housing 12, thus penetrating the skin of the person or intravenous administration set being injected. Continued pressing of the upper section of the actuator 26 presses the upper arm 86 of the actuator 26 against the lower arm 88 extending upright from the floor 82. An inner flange 92 of the collar 84 squeezes inward to lock into notches 90 of the upper arm 86 and locks onto the upper arm 86, as shown in FIG. 9, thereby securing the collar 84 to the upper arm 86. The needle 24 includes a central passageway to its sharp tip. A notch 94 extends through the sidewah of the needle 24 for communication with the central passageway in the needle 24. When the actuator 26 has been depressed to the position shown in FIG. 9, the needle notch 94 of the injection needle 24 is in alignment with the channel 56 in the housing 12 to provide a conduit for the drug solution to flow out of the syringe 16 and through the injection needle 24 into the patient being administered. In particular, when the notch 94 becomes aligned with the channel 56, the pressure of the drug solution in the barrel 50 of the syringe 16 is released, and the syringe spring 44 pushes the syringe's piston 62 downward to force the drug solution through the channel 56 and the communicating injection needle 24 into the patient.

FIGS. 10 and 11 illustrate the relative positions of the plunger 46 upon completion of the drug solution delivery. To that end as can be seen at that time the syringe plunger 47 is extended through the barrel 50 so that its proximal end is nearly flush with the handle or cap 48 indicating that the delivery has been completed. This relation between the rod 47 and the handle 48 provides the benefit of indicating to the patient that delivery has ended, which can be determined from feeling the rod 47 and handle 48. Accordingly, visual contact for injection of the drug is not required.

Upon the end of delivery, the user stops pressing the upper section of the actuator 26. This action causes the spring 76 to bias the upper section back to a position like that of FIG. 7, whereupon the injection needle retracts into the housing, as shown in FIGS. 12 and 13. When the actuator 26 is forced upward by the injector spring 76, the collar 84 is pulled up with the upper arm 86 off of the lower arm 88. The collar 84 is arranged so that when it is pulled off the lower arm its distal end contracts radially inward toward the injection needle 24 as the lower end of the collar 84 clears the top edge of the lower arm 88. As a result, the collar 84 rests on the upward lip of the lower arm 88. Any subsequent downward force applied to the actuator 26 will not move the upper arm 86, thus preventing the re-extension of the injection needle 24.

Referring to FIGS. 14 and 15, there is shown at 100 a reconstitution and injection system constructed in accordance with another preferred embodiment of this invention. The system 100 includes a housing 102 formed of any suitable material (e.g., plastic or metal) having a first recessed port or barrel 104 for receiving a syringe plunger 106 and handle 108, and a second recessed port 110 for receiving a drug cartridge or vial 112 (conventional or otherwise).

The housing 102 also includes a third recessed port 114 for receiving an injection needle 116. The injection needle 116 is hollow and serves as a means for delivering the reconstituted drug to the patient. The injection needle 116 preferably has a penetration length of about 7 mm. However, the penetration length of the injection needle 116 is not limited to a length of 7 mm since it is understood that the injection needle may be any length and thickness (e.g., 26 gage) sufficient to penetrate the skin and deliver the drug compound.

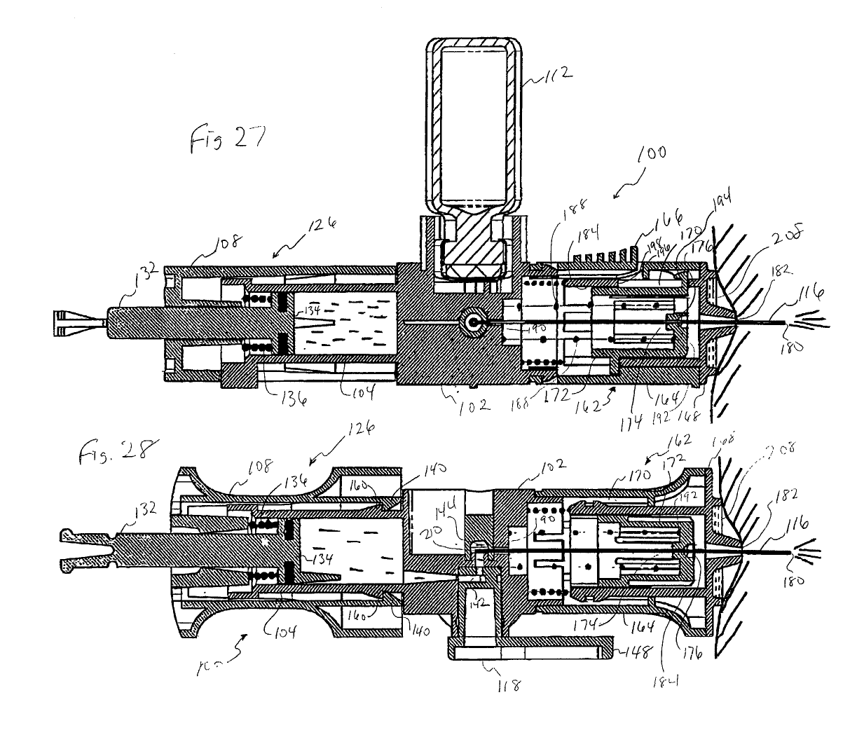

A spring-loaded actuator 162 is coupled to the third recessed port 114 for moving the injection needle 116 from the housing 102 into the drug receiver (e.g., patient or intravenous administration set) for injection, as shown in FIGS. 27 and 28 (to be described later). The housing 102 also includes a pathway lever 118 having a cylindrical stem 120 located in a pathway bore 122 of the housing 102 for controlling the flow of a liquid or drug solution within the housing 102 (as will be described later). A vial needle 124 is located within the housing 102 in the second recessed port 110 for communication with the vial 112.

In this embodiment, the barrel 104 forms a tubular section of the syringe 126, which also includes the plunger 106 and the handle 108. In this exemplary embodiment, the syringe 126 is empty and the diluent is provided in the vial 112. Alternatively, the syringe 126 may be refilled with a diluent and the vial 112 may contain a lyophilized drug or compound concentrate, as shown in the embodiment of FIG. 1. In either case, as shown in FIG. 16, the vial 112 is pressed into the second recessed port 110 until the vial needle 124 penetrates a rubber stopper 128 (FIG. 16) of the vial 112 that seals the vial 112, such that the vial needle 124 extends into the interior of the vial 112.

The vial needle 124 is hollow and acts as a passageway through which gas or fluid from the syringe 126 may flow into the vial 112 when the syringe's plunger 106 is depressed. To that end, as shown in FIG. 16, the vial needle 124 communicates with a first channel 130 in the housing 102 that extends to the distal end of the syringe 126. As shown in FIGS. 15 and 16, the first channel 130 is formed along the periphery of the cylindrical stem 120 of the pathway lever 118 that extends into the pathway bore 122 of the housing 102. Upon insertion of the vial 112 into the second recessed port 110, the vial needle 124 pierces the rubber stopper 128 of the vial 112, thus opening communication between the vial 112 and the syringe 126. Pressing the syringe's plunger 106, like shown in FIG. 17a, causes the gas or fluid within the barrel 104 to flow out of the syringe 126 into the first channel 130 and through the vial needle 124 into the vial 112.

When desired, fluid access from the vial 112 to the syringe 126 is prevented by rotating the pathway lever 118 such that the cylindrical stem 120 rotates to block the outlet of the first channel 130 serving as a pathway to the vial needle 124. As with the system 10 described above and shown in FIGS. 1 through 13, when liquid or diluent is present in the syringe 126, it is preferable that the pathway lever 118 is positioned so that its cylindrical stem 120 precludes communication between the vial 112 and syringe 126 until the vial 112 is also inserted into the housing 102. In this manner, the liquid or diluent in the syringe 126 is not spilled through the first channel 130 into the second recessed port 110.

Once both the syringe 126 and vial 112 are secured into place within the housing 102, for example, as shown in FIGS. 15-17, the pathway lever 118 can be rotated to place the interior of the barrel 104 in fluid communication with the interior of the vial 112. The syringe 126 and vial 112 are secured in place when their interiors are in fluid communication with the cylindrical stem 120 of the pathway lever 118. For example, the syringe 126 is secured in place for purposes of fluid communication with the cylindrical stem 120 by inserting the syringe plunger 106 into the barrel 104 such that fluid in the barrel 104 between the plunger 106 and the distal end of the syringe 126 preferably exits through the distal end towards the cylindrical stem 120. The vial 122 is secured in place by inserting the vial 112 into the second recessed port 110 such that the vial needle 124 pierces the rubber stopper 128 of the vial 112. In this example, the second recessed port 110 includes ribs 113 extending radially inward from an inner wall of the second recessed port 110. When the vial 112 is inserted into the second recessed port 110, the vial 112 frictionally engages the ribs 113 thereby further securing the vial 112 in place within the second recessed port 110. It is understood that the ribs 113 are one of many alternative approaches that could frictionally engage and help secure the vial. The second recessed port 110 or the vial 112 could be modified such that the vial 112 is securely held within the second recessed port 110 as desired.

In this exemplary embodiment, the housing 102 includes the barrel 104 of the syringe 126. Alternatively, the syringe 126 may be a standard or conventional syringe 126 which is coupled to the housing 102, for example, via locking tabs such as shown in the exemplary system 10 shown in FIG. 1. As noted above, the syringe 126 includes the plunger 106 having a rod 132 and a piston 134 at the distal end of the plunger 106. The plunger 106 is slidingly located within the barrel 104, which, as shown in FIG. 16, is empty. The rod 132 is slidingly located within the handle 108 and includes fingers 133 (FIG. 15) that are radially biased outward towards the handle 108. The fingers 133 are snap fitted into notches 109 (FIGS. 24 and 26) of the handle 108 and can be released from the notches 109 towards the proximal end of the handle as desired to slide the handle 108 beyond the rod 132 and along the barrel 104 toward the housing 102. A helical compression syringe spring 136 (FIG. 24) is provided about the plunger 106 between the handle 108 and the piston 134.

The piston 134 includes an “O” ring 138 (FIG. 16) formed of an elastomeric material and having an outer diameter slightly greater than the inner diameter of the barrel 104 to form a sliding seal therewith so that no fluid can gain egress through the interface of the piston 134 and the barrel 104. Alternatively, the piston 134 can be formed of an elastomeric material and have an outer diameter slightly greater than the inner diameter of the barrel 104. Either construction is preferred because the piston 134 makes sliding frictional engagement with the inner wall of the barrel 104 for pushing or pulling a solution out of or into the barrel 104. The handle 108 is in the form of a cap having a larger diameter than the barrel 104. A pair of clips 140 (FIGS. 15 and 24) extend inward from the distal end of the handle 108 for connecting to the barrel 104 at outwardly extending tabs 160 of the barrel 104, as will be described below.

As shown in FIG. 16, the cylindrical stem 120 of the pathway lever 118 also includes a second channel 142 that provides communication from the syringe 126 to an injection needle 116. The second channel 142 is made up of a radial bore 122 that extends from a circumferential edge of the cylindrical stem 120 to its central axis, and a longitudinal bore that continues along the central axis to its distal end into an injection chamber 144 (FIG. 24) of the housing 102, as will be described in greater detail below. The second channel 142 is rotatably engageable with the distal end of the syringe 126, and is always in communication with the injection chamber 144. In this exemplary embodiment, the housing 102 provides the communication between the syringe 126, drug vial 112 and injection needle 116.

FIGS. 16-20 generally illustrate the exemplary steps for reconstitution of the drug compound in accordance with a preferred method of use of the system 100. To that end, as shown in FIG. 16, the syringe's plunger 106 is initially in a retracted position and the vial 112 contains a diluent. The vial 112 is maintained in its recess 110 by friction, and the vial 112 can be replaced by other vials 112 as needed to provide the desired compound for injection, as will be described later. The pathway lever 118 is in a first position, allowing communication between the syringe 126 and the vial 112 via the first channel 130.

In FIG. 17, the syringe's plunger 106 is depressed into the barrel 104 until air in the barrel 104 has been moved from the syringe 126 into the vial 112. In this condition, the plunger 106 of the system 100 is fully depressed into the barrel 104 of the syringe 126 to push the air into the vial 112. This action increases the pressure in the vial 112 and allows for easier retraction of the plunger 106.

In FIG. 18, the plunger 106 and the handle 108 are pulled back which causes the diluent to move from the drug vial 112 into the syringe 126. In accordance with one preferred embodiment, the syringe 126 is translucent and includes a series of visual indications as a scale thereon, as understood by a skilled artesian, to enable an accurate measurement of the level of liquid drawn into the barrel 104 of the syringe 126 to be made. Therefore, aspiration of the diluent is measured by visual observation of the scale. If a user gets more diluent than desired, it is possible to push the excess diluent back to the vial 112 or even to start the process again by pushing all of the diluent into the vial 112. Accordingly, a user can adjust the position of the piston 134 within the barrel 104 to the appropriate level of liquid (e.g., diluent, solution).

As noted above, the vial 112 can be replaced with any other vial 112a containing a lyophilized drug or solution, for mixing with the diluent or compound. In this manner, several drug vials 112 can be used for reconstitution of a drug compound by replacing one drug vial 112 with another and mixing the contents of each drug vial 112 with the drug solution until the desired compound is mixed for injection. As shown in FIG. 19, the diluent vial 112 is replaced by a drug vial 112a by removing the diluent vial 112 from the second recessed port 110 and pressing the drug vial 112a into the second recessed port 110 until the vial needle 124 penetrates the rubber stopper 128 of the drug vial 112a that seals the vial 112a, such that the vial needle 124 extends into the interior of the vial 112a.

As shown in FIG. 20, the plunger 106 is depressed into the barrel 104 until the diluent has been moved from the syringe 126 into the vial 112a. The plunger 106 is fully depressed into the barrel 104 of the syringe 126 to push the fluid (e.g., diluent, drug solution) into the drug vial 112a for mixing with the lyophilized or powdered drug compound. The system 100 is swirled by, for example, a user to further ensure complete reconstitution of the drug/diluent solution.

FIG. 21 illustrates the relative positions of the plunger 106 in the syringe 126 upon aspiration of the drug compound. As shown, the plunger 106 and handle 108 are pulled back which causes the reconstituted solution to move from the drug vial 112a into the syringe 126. As noted above, a user can adjust the position of the piston 134 to the desired level of solution for injection by seeing the position of the piston relative to the scaled lines on the housing.

Once the plunger 106 moves the desired amount of reconstituted solution from the drug vial 112a into the syringe 126, the pathway lever 118 is rotated about its axis. This causes the cylindrical stem 120 having the channel 130 to rotate about that axis within the pathway bore 122 of the housing 102 to terminate the liquid communication between the drug vial 112a and the syringe 126, as shown in FIGS. 22-24. The pathway lever 118 includes an interlocking arm 146 and a pointer arm 148. Both arms extend radially outward from the axis of the pathway lever 118. The interlocking arm 146 has an extension 150 at its distal end projecting inward toward the housing 102. Upon rotation of the pathway lever 118, the interlocking arm 146 rotates until its extension 150 abuts against a blocking edge 152 of the second recessed port 110. In this location, the extension 150 slides into a channel region 154 of the vial 112a to prevent the ejection of the vial 112a from its location in the second recessed port 110. By locking the vial 112a in this position, the interlocking arm 146 provides the added safety measure of keeping the vial needle 124 in the vial 112a so that the vial needle 124 is not exposed while the drug is being injected into the patient or during subsequent handling of the system (e.g., after injection when the system 100 is being thrown away).

As mentioned above, the first channel 130 permits fluid communication between the drug vial 112a and the syringe 126 as part of the preparation for injection. Once the preparations (e.g., reconstitution, getting the desired concentration, titration) are completed, then fluid communication between the drug vial 112a and the syringe 126 should be terminated, and the drug vial 112a should preferably be secured and locked to the housing. As shown in FIG. 23, when the pathway lever 118 is rotated into its locked position, the cylindrical stem 120 is also rotated, which disconnects the communication between the drug vial 112a and syringe 126. In addition, this action opens the communication between the syringe 126 and the injection needle 116 via the second channel 142, and further, locks the drug vial 112a in the second recessed port 110.

FIGS. 24 through 30 illustrate the various steps for pressurization and injection of the drug compound in accordance with a preferred method of use of the system 100. To that end, as shown in FIG. 24, the plunger 106 is shown in its retracted position and the desired amount of reconstituted solution is in the barrel 104 of the syringe 126. The reconstituted solution communicates through the second channel 142 into the injection chamber 144 defined by an injection septum 156 (FIGS. 15, 24 and 26b). The injection septum 156 is inserted into the housing 102 through a cylindrical aperture 158 in the housing 102 opposite the pathway bore 122. As shown in FIGS. 15, 24 and 26b, the injection septum 156 is a cylindrical body with a cup shaped distal end that defines the injection chamber 144. A central bore 186 extends through the housing 102 and the injection septum 156. The bore 186 intersects the injection chamber 144 which is in communication with the interior of the syringe 126 via the second channel 142 in the pathway lever 118. The injection needle 116 is slidingly engaged within the bore 186. Prior to injection, the injection needle 116 blocks access to its hollow interior, thereby confining the solution about the injection needle 116 within the injection chamber 144 so that no drug solution can escape via the syringe 126.

Once the pathway lever 118 is rotated to block communication between the syringe 126 and the vial 112a, the system 100 is ready to have its handle 108 depressed, as will be described hereinafter, to place the drug solution under pressure. In particular, as shown in FIGS. 22, 25a, 25b, 26a and 26b, the handle 108 is depressed toward the housing 102. Since the drug solution is locked within the barrel 104, the incompressibility of the solution acts as a stopper to the piston 134. Depressing the handle 108 releases the fingers 133 from the notches 109, thereby disconnecting the rod 132 from the handle 108. The handle 108 moves forward, causing the syringe spring 136 along the rod 132 to compress between the piston 134 and the handle 108, thus placing the drug solution in the barrel 104 under pressure. Clips 140 on the interior wall of the handle 108 engage and lock about outwardly extending tabs 160 on the outer wall of the barrel 104 (FIG. 26b) to maintain the compression of the syringe spring 136 until the drug solution in the barrel 104 of the syringe 126 is released by the action of the drug injector subsystem.

The drug injector subsystem is best seen in FIGS. 25a, 25b, 26a and 26b. FIGS. 25a and 26a are longitudinal and transverse sectional views, respectively, taken at right angles of each other, showing the subsystem in its “pre-injection position”. FIG. 25b is a partial longitudinal sectional view of the system of FIG. 25a. FIG. 26b is a partial transverse sectional view of the system of FIG. 26a. The drug injection subsystem basically comprises an actuator 162 including an actuator housing 164 having an injection latch 166 that triggers movement of the injection needle 116 through a shield 168 and into the patient. The actuator housing 164 has a hollow cylindrical axial channel 170 that receives the shield 168 and an injection needle hub 172 that is slidingly engaged within the shield 168. The injection needle hub 172 has a cup shaped upper section 174 having a centrally located aperture 176. The aperture 176 mounts about the injection needle 116 and holds the needle 116 in a first position such that the proximal end 178 of the injection needle 116 in the central bore 186 extends beyond (outside) the injection chamber 144, and the distal sharp end 180 of the injection needle 116 extends close to but not beyond an opening 182 in the shield 168 as shown in FIGS. 25a, 25b and 26a. The shield 168 includes a cup shaped proximal section 184 that slidingly receives the injection needle hub 172. When assembled, the actuator housing 164 slidingly receives the shield 168 at its distal end, and the shield 168 slidingly receives the injection needle hub 172 at its proximal end.

The actuator housing 164, which is snap-fitted to the housing 102 (FIG. 25a), also encloses two helical compression springs. The first spring is a needle hub spring 188 that is located within the interior of the needle hub 172 and housing 102 immediately adjacent the inner surface of the hub 172 between an inner back wall 190 of the housing 102 and an inner forward wall 192 of the hub 172. The needle hub spring 188 is held in a compressed state (FIGS. 24-26a) while a hub latch 194 extending from a distal peripheral edge of the hub 172 abuts a holding wall 196 of the actuator housing 164. The hub latch 194 also communicates with the injection latch 166 such that the needle hub spring 188 is released when the injection latch 166 is depressed to push the hub latch 194 inward which frees itself from the holding wall 196 of the actuator housing 164.

A locking edge 198 of the cylindrical wall of the shield 168 also abuts the hub latch 194 when the shield 168 is in its pre-injection position as is best seen in FIG. 25b. When the locking edge 198 abuts the hub latch 194, the hub latch 194 is inhibited from being pushed inward by the injection latch 166. Therefore, in this pre-injection position, the shield 168 abuts the hub latch 194 so that the injection latch 166 will not accidentally release the needle hub spring 188 and force the injection needle 116 through the shield 168. The second helical compression spring within the actuator housing 164 is a shield spring 200 located within the interior of the housing 102 immediately adjacent an inner surface of a sidewall 202 of the housing 102 between a proximal end 204 of the shield 168 and outer back wall 206 of the housing 102.

When the handle 108 is locked against the housing 102, placing the drug solution under pressure, as shown in FIGS. 25a and 26a, the system 100 is ready for injecting a patient. To that end, as shown in FIGS. 27 and 28, a front side 208 of the shield 168 is positioned so that it faces the patient and is pressed down onto the injection site. This pressure causes the shield 168 to retract and pushes the locking edge 198 of the shield 168 towards the housing 102. This movement of the shield 168 leaves a gap between the locking edge 198 and the hub latch 194, such that the hub latch 194 can be pressed inward by the injection latch 166 until the hub latch 194 can slide under the holding wall 196 of the actuator housing 164. The injector latch 166 is pushed down which presses the hub latch 194 and releases it from the holding wall 196. Upon this release, the needle hub spring 188 longitudinally expands and biases the needle hub 172 toward the distal end of the shield 168. The hub 172, which is holding the injection needle 116, pushes the distal sharp end 180 of the needle 116 through the opening 182 of the shield 168, thus instantly penetrating the skin of the person or intravenous administration set being injected. As noted above, the penetration length of the needle 116 is preferably about 7 mm, although any length that penetrates the skin (or intravenous administration set) and delivers the drug solution is sufficient.

The injection needle 116 includes a central passageway extending from an opening 210 at its proximal end 178 to an opening at its distal sharp end 180. When the injection needle 116 is pushed by the needle hub 172 to extend the distal sharp end 180 of the injection needle 116 beyond the shield 168, as shown in FIGS. 27 and 28, the proximal end opening 210 of the injection needle 116 is in communication with the interior of the injection chamber 144 in the housing 102. The central passageway of the needle 116 provides a conduit for the drug solution to flow out of the syringe 126 and through the injection needle 116 into the patient or intravenous administration set being administered. In particular, when the opening 210 comes into communication with the interior of the injection chamber 144, the pressure of the drug solution in the barrel 104 of the syringe 126 is released. This causes the syringe spring 136 to instantly push the syringe's piston 134 downward to force the drug solution through the second channel 142 and the communicating injection needle 116 into the patient.

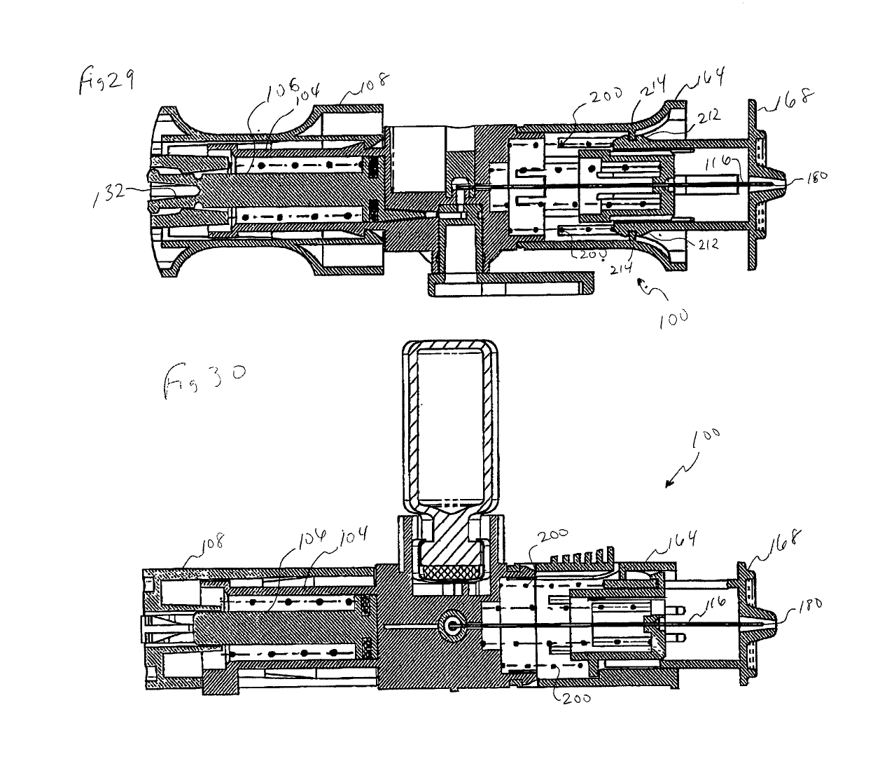

FIGS. 29 and 30 illustrate the relative positions of the plunger 106 and shield 168 upon completion of the drug solution delivery. To that end, as can be seen at that time, the syringe plunger 106 is extended through the barrel 104 so that its proximal end is nearly flush with the handle 108, indicating that the delivery has been completed. This relation between the plunger 106 and the handle 108 provides the benefit of indicating to the patient that drug delivery has been completed, which can be determined from feeling the proximal ends of the rod 132 and handle 108. Accordingly, in both examples of the embodiments discussed herein, tactile confirmation of an injection is provided and the user need not look at the device or the injection site to confirm a successful injection.

Upon the end of delivery, the user stops pressing the shield 168 against the injection site. This action causes the shield spring 200 to bias the shield 168 forward to cover the distal sharp end 180 of the injection needle 116. The shield 168 includes latches 212 that abut and snap-fit about inwardly extending tabs 214 of the actuator housing 164 to lock the shield 168 in its extended position (FIGS. 29 and 30). Any subsequent force applied to the system 100 will not move the shield 168, thus preventing the re-exposure of the injection needle 116.

As should be appreciated from the foregoing, the reconstitution and injection systems of the preferred embodiments provide a safe and efficient approach to mixing and injecting a drug compound into a patient. The reconstitution and injection system requires no air to push liquids in any sequence, other than possibly to prevent effects of vacuum. The system is designed to meet different standard syringes, and a syringe refilled with diluent can be easily adopted for use.

The reconstitution and injection system allows for accurate titration in measurement of the amount of drug compound to be injected, and provides an approach for fixing mistakes of overdose or air bubbles. A skilled artisan can readily understand that this approach allows for the implementation of several vials for the same injection, as vials can be replaced while the drug compound is locked within the barrel of the syringe, or as the housing is adapted to receive additional vials. Since the end of delivery indication is clear, no eye contact is required for indication of the end of delivery, thus making delivery easier when the user can not see the injection area.

It should be apparent from the aforementioned description and attached drawings that the concept of the present application may be readily applied to a variety of preferred embodiments, including those disclosed herein. For example, other retractors, such as elastomeric o-rings or compressed gas, may be used in place of the helical compression springs disclosed herein to bias the plunger, piston, hub, shield, pushing member or actuators, as readily understood by a skilled artesian. Since the embodiments of the system shown in the figures include all four subsystems (e.g., reconstitution, pressurization, transfer and injector), it is understood that any subsystem of one embodiment would work alternatively in other embodiments of the system. Moreover, for example, the pressurization subsystem shown in the exemplary embodiment shown in FIGS. 1-13 would also work as an alternative to the pressurization subsystem shown in FIGS. 14-30, and vice versa.

It is further appreciated that the present invention may be used to deliver a number of drugs. The term “drug” used herein includes but is not limited to peptides or proteins (and memetics thereof), antigens, vaccines, including DNA vaccines, hormones, analgesics, anti-migraine agents, anti-coagulant agents, medications directed to the treatment of diseases and conditions of the central nervous system, narcotic antagonists, immunosuppressants, agents used in the treatment of AIDS, chelating agents, anti-anginal agents, chemotherapy agents, sedatives, anti-neoplastics, prostaglandins, antidiuretic agents and DNA or DNA/RNA molecules to support gene therapy.

Typical drugs include peptides, proteins or hormones (or any memetic or analogues of any thereof) such as insulin, calcitonin, calcitonin gene regulating protein, atrial natriuretic protein, colony stimulating factor, betaseron, erythropoietin (EPO), interferons such as α, β or γ interferon, somatropin, somatotropin, somastostatin, insulin-like growth factor (somatomedins), luteinizing hormone releasing hormone (LHRH), tissue plasminogen activator (TPA), growth hormone releasing hormone (GHRH), oxytocin, estradiol, growth hormones, leuprolide acetate, factor VIII, interleukins such as interleukin-2, and analogues or antagonists thereof, such as IL-1ra, thereof; analgesics such as fentanyl, sufentanil, butorphanol, buprenorphine, levorphanol, morphine, hydromorphone, hydrocodone, oxymorphone, methadone, lidocaine, bupivacaine, diclofenac, naproxen, paverin, and analogues thereof; anti-migraine agents such as sumatriptan, ergot alkaloids, and analogues thereof; anti-coagulant agents such as heparin, hirudin, and analogues thereof; anti-emetic agents such as scopolamine, ondansetron, domperidone, metoclopramide, and analogues thereof; cardiovascular agents, anti-hypertensive agents and vasodilators such as diltiazem, clonidine, nifedipine, verapamil, isosorbide-5-mononitrate, organic nitrates, agents used in treatment of heart disorders, and analogues thereof; sedatives such as benzodiazepines, phenothiozines, and analogues thereof; chelating agents such as deferoxamine, and analogues thereof; anti-diuretic agents such as desmopressin, vasopressin, and analogues thereof; anti-anginal agents such as nitroglycerine, and analogues thereof; anti-neoplastics such as fluorouracil, bleomycin, and analogues thereof; prostaglandins and analogues thereof; and chemotherapy agents such as vincristine, and analogues thereof, treatments for attention deficit disorder, methylphenidate, fluoxamine, Bisolperol, tactolimuls, sacrolimus and cyclosporin.

Without further elaboration, the foregoing will so fully illustrate the invention that others may, by applying current or future knowledge, readily adapt the same for use under various conditions of service. What is claimed is:

1. A drug delivery device, comprising:
a syringe having a barrel, a plunger, a handle and a pump, said barrel having a drug reservoir therein, said reservoir being arranged to have a liquid therein, said plunger slidingly located within said barrel and coupled to said plunger, and said pump located between said plunger and said handle; and
a first channel arranged for selectively providing fluid communication between said drug reservoir and a hollow needle,
said pump being arranged to drive the liquid from the drug reservoir through said needle via said first channel upon said fluid communication between said drug reservoir and said needle to deliver the liquid to a site.

2. The drug delivery device of claim 1, wherein said pump comprises a compression spring.

3. The drug delivery device of claim 1, wherein said handle includes clips arranged to couple said handle to said barrel to at least temporarily place said pump in a compressed state between said plunger and said handle.

4. The drug delivery of claim 1, wherein said pump biases said plunger to drive the liquid from said drug reservoir through said needle via said first channel upon said fluid communication between said drug reservoir and said needle.

5. The drug delivery device of claim 1, additionally comprising a vial receptacle arranged to receive a vial and a housing, and wherein said first channel is located in said housing, said housing including a second channel arranged for selectively providing fluid communication between said drug reservoir and the vial in said vial receptacle.

6. The drug delivery device of claim 5, wherein said vial receptacle includes a spiking needle having a first end communicating with said second channel and a second end arranged for selectively providing fluid communication with the interior of the vial in said vial receptacle.

7. The drug delivery device of claim 5, wherein said device further comprises a pathway controller arranged for inhibiting fluid communication between said drug reservoir and the interior of the vial.

8. The drug delivery device of claim 7, wherein said pathway controller includes a stem slidingly engaged within said second channel to permit fluid communication between said drug reservoir and the interior of the vial when said pathway controller is in a first position and to inhibit fluid communication between said drug reservoir and the interior of the vial when said pathway controller is in a second position.

9. The drug delivery device of claim 7, wherein said pathway controller includes a stem rotatably engaged within said housing and adjacent said second channel to permit fluid communication between said drug reservoir and the interior of the vial when said pathway controller is in a first position and to inhibit fluid communication between said drug reservoir and the interior of the vial when said pathway controller is in a second position.

10. The drug delivery device of claim 9, wherein said pathway controller comprises an arm extending from said pathway controller such that when said pathway controller is in the second position said arm abuts the vial to inhibit removal of the vial from said housing and said stem allows fluid communication between said drug reservoir and said injection needle.

11. The drug delivery device of claim 5, wherein said housing includes an interlocking arm that releasably secures the vial to said housing.

12. The drug delivery device of claim 1, further comprising an actuator coupled to said housing, said actuator arranged for aligning an opening in said needle with said first channel to permit the fluid communication between said drug reservoir and said needle.

13. The drug delivery device of claim 12, wherein said actuator includes a hub that holds said needle, said hub being arranged for moving said needle from a first position where said opening in said needle is not in fluid communication with said drug reservoir to a second position where the opening in said needle is in fluid communication with said drug reservoir.

14. The drug delivery device of claim 13, wherein said needle comprises a tip that is concealed by said drug delivery device when said needle is in said first position and is exposed when said needle is in said second position.

15. The drug delivery device of claim 13, further comprising a second pump located between said housing and said actuator, said second pump being arranged to bias said actuator such that said needle is concealed by said drug delivery device upon completion of the delivery of the liquid to the injection site.

16. The drug delivery device of claim 15, wherein said second pump comprises a compression spring.

17. The drug delivery device of claim 13, wherein said actuator includes an actuator housing and a shield, said actuator housing having a first end coupled to the housing and a second end adjacent said shield, said shield arranged to conceal said tip of said needle when said needle is in said first position.

18. The drug delivery device of claim 17, further comprising a second pump located between said housing and said hub, said second pump being arranged to bias said hub to push said needle from said first position to said second position.

19. The drug delivery device of claim 18, wherein said second pump is a helical compression spring.

20. The drug delivery device of claim 18, further comprising a latch coupled to said actuator and said hub, said latch being arranged to activate said second pump to push said needle to said second position.

21. The drug delivery device of claim 18, further comprising a third pump located between said housing and said shield, said third pump being arranged to bias said shield such that said needle is concealed by said drug delivery device upon completion of the delivery of the liquid to the injection site.

22. The drug delivery device of claim 21, wherein said second and third pumps comprise compression springs.

23. The drug delivery device of claim 1, wherein said needle is an injection needle.

24. The drug delivery device of claim 23, wherein said injection needle has a penetration length of about 7 mm.

25. The drug delivery device of claim 1, wherein the site of delivery is to or through the skin of a mammal.

26. The drug delivery device of claim 1, wherein said needle has a gage of about #26.

27. A syringe, comprising:
a barrel having a drug reservoir therein, said drug reservoir being arranged to hold a liquid therein;
a plunger slidingly engaged within said barrel;
a handle coupled to said plunger; and
a pump located between said plunger and said handle, said drug reservoir and a needle being arranged to be selectively placed in fluid communication with each other, said pump arranged for driving the liquid from said drug reservoir through said needle upon said fluid communication between said drug reservoir and said needle for delivery of the liquid to a site.

28. The syringe of claim 27, wherein said pump comprises a compression spring.

29. The syringe of claim 27, wherein said handle includes at least one clip arranged to couple said handle to said barrel to at least temporarily place said pump in a state of potential energy between said plunger and said handle.

30. The syringe of claim 27, wherein said pump is arranged to bias said plunger to drive the liquid from said drug reservoir through said needle upon fluid communication between said drug reservoir and said needle.

31. The syringe of claim 27, wherein said plunger comprises a piston coupled to a rod, said piston forming a sliding seal within said barrel, said rod slidingly engaged within said handle and having a proximal end arranged to snap-fit said handle.

32. The syringe of claim 27, wherein said needle is an injection needle.

33. The syringe of claim 27, wherein the site of delivery is to or through the skin of a mammal.

34. The syringe of claim 27, wherein said needle has a penetration length of about 7 mm.

35. The syringe of claim 27, wherein said needle has a gage of about #26.

36. A mixing device coupled to a syringe, the syringe having a drug reservoir therein, said mixing device comprising:
a first port arranged for receiving the syringe, the syringe having a barrel coupled to or integral with said first port, the barrel having a drug reservoir therein, the drug reservoir being in fluid communication with said first port;
a second port arranged for receiving a vial, the vial having an interior in fluid communication with said second port;
a first channel arranged for selectively communicating said first port with said second port; and
a pathway controller in fluid communication with the first channel and between said first and second ports, said pathway controller arranged for permitting the fluid communication between said first and second ports when said controller is in a first position and for inhibiting the fluid communication between said first and second ports when said controller is in a second position.

37. The mixing device of claim 36, wherein said second port includes a spiking needle having a first end communicating with said first channel and a second end arranged for selectively providing fluid communication with the interior of the vial in said vial receptacle.

38. The mixing device of claim 36, wherein said pathway controller comprises a stem slidingly engaged within said first channel.

39. The mixing device of claim 36, wherein said pathway controller comprises a stem rotatably engaged within said mixing device and adjacent said first channel.

40. The mixing device of claim 39, wherein said pathway controller comprises an arm extending from said pathway controller such that when said pathway controller is in said second position, said arm abuts the vial to inhibit removal of the vial from said mixing device.

41. The mixing device of claim 36, further comprising:
a third port arranged for receiving an actuator, said actuator comprising a needle, and
a second channel between said first port and said third port for communicating said drug reservoir with said needle.

42. The mixing device of claim 41, wherein said actuator includes a hub that holds the needle, said hub being arranged for moving said needle from a first position where the opening in said needle is not in fluid communication with said second channel to a second position where the opening in said needle is in fluid communication with said second channel.

43. The mixing device of claim 42, wherein said needle is an injection needle.

44. A drug delivery apparatus, comprising:
an actuator having a hub and a hollow needle, said hub holding said needle, said needle having a tip at its distal end and an opening proximal to the tip; and
a housing coupled to said actuator, said housing including a channel arranged to selectively provide fluid communication between a liquid reservoir holding a liquid and said needle, wherein
said hub is arranged for moving said needle from a first position where said opening in said needle is not in fluid communication with said channel to a second position where said opening in said needle is in fluid communication with said channel to deliver a liquid from the liquid reservoir.

45. The drug delivery apparatus of claim 44, wherein said needle is an injection needle.

46. The drug delivery apparatus of claim 45, wherein said injection needle has a penetrating length of about 7 mm.

47. The drug delivery apparatus of claim 45, wherein said injection needle comprises a tip, and said tip of said injection needle is concealed by said drug delivery apparatus when said injection needle is in said first position and is exposed when said injection needle is in said second position.

48. The drug delivery apparatus of claim 47, wherein said injection needle has a penetration length of about 7 mm.

49. The drug delivery apparatus of claim 44, wherein said actuator further comprises a pump located between said housing and said hub, said pump being arranged to bias said hub such that said tip of said needle is concealed by said drug delivery apparatus upon completion of the delivery of the liquid.

50. The drug delivery apparatus of claim 49, wherein said pump comprises a compression spring.

51. The drug delivery apparatus of claim 44, wherein said actuator further includes an actuator housing and a shield, said actuator housing having a first end coupled to said housing and a second end adjacent said shield, said shield being arranged to conceal said needle when said needle is in said first position.

52. The drug delivery apparatus of claim 51, wherein said actuator further comprises a first pump located between said housing and said hub, said first pump being arranged to bias said hub to push said needle from said first position to said second position beyond said shield.

53. The drug delivery apparatus of claim 52, wherein said first pump comprises a compression spring.

54. The drug delivery apparatus of claim 52, wherein said actuator further includes a second pump located between said housing and said shield, said second pump being arranged to bias said shield and push said shield away from said housing such that said needle is concealed by said shield upon completion of the delivery of the liquid.

55. The drug delivery apparatus of claim 54, wherein said first and second pumps comprise compression springs.

56. The drug delivery apparatus of claim 52, further comprising a latch coupled to said actuator and said hub, said latch being arranged to activate said first pump to push said needle to said second position.

57. A method of ejecting a fluid from a barrel of a syringe having a handle, a barrel, a plunger and a needle, said barrel having the fluid therein, said needle being hollow, the method comprising:
securing said handle of the syringe to said barrel to bias said plunger against said fluid in said barrel placing said fluid under pressure; and
actuating a movement of said needle to place the hollow interior of said needle in fluid communication with said fluid whereupon said plunger automatically pushes said fluid through said needle.

58. A method of mixing a drug in a vial with a liquid from a reservoir, comprising:
(a) establishing fluid communication between a control valve and the interior of said vial, the interior of said vial containing a drug;
(b) adjusting said control valve to selectively establish fluid communication between said liquid in said reservoir and the interior of said drug vial via said control valve, said reservoir being in fluid communication with said control valve; and
(c) transferring said liquid to the interior of said drug vial, said liquid mixing with said drug to form a drug compound.

59. The method of claim 58, further comprising:
transferring said drug compound to said reservoir;
adjusting said control valve to cease fluid communication between said reservoir and the interior of said drug vial;
terminating fluid communication between said control valve and the interior of said drug vial;
establishing fluid communication between said control valve and the interior of another drug vial, the interior of said other drug vial containing another material;
adjusting the control valve to establish fluid communication between the reservoir and the interior of the other drug vial; and
transferring said drug compound to the interior of said other drug vial, the drug compound mixing with said other material to form a reconstituted drug compound.

60. The method of claim 58, prior to step (a), further comprising:
adjusting said control valve to establish fluid communication between said reservoir and the interior of a diluent vial having the liquid therein;
aspirating the liquid into said reservoir;
adjusting said control valve to cease fluid communication between said reservoir and the interior of said diluent vial; and
terminating fluid communication between said control valve and the interior of said diluent vial.

61. A method of ejecting a liquid from a barrel of a syringe, said syringe comprising a needle, a plunger located within the barrel and having a stem and a handle, comprising:
depressing said handle of said plunger to cause said plunger to slide within the barrel to couple said handle to said barrel to place said liquid under pressure; and
actuating a movement of said needle within said syringe for insertion into an injection site, the movement of the needle establishing fluid communication between an interior of the needle and said liquid which releases the pressure of said liquid in said barrel and enables said plunger to push said liquid through the needle to the injection site.

62. The method of claim 61, wherein a compression pump is associated with said syringe, and depressing the handle of said plunger causes said compression pump to pressurize said liquid.

63. The method of claim 62, wherein said compression pump comprises a spring, and said method comprises compressing said spring.

64. A method of ejecting a liquid from a barrel of a syringe, said syringe comprising a hollow needle, a plunger, and a compression pump, said method comprising:
applying a bias against said plunger in communication with said liquid to place the liquid under pressure; and
actuating a movement of said needle within said syringe for insertion into an injection site, the movement of said needle establishing fluid communication between the hollow interior of said needle and said liquid which releases the pressure of said liquid in said barrel and enables said compression pump to push said plunger within said barrel and force said liquid through the needle.

65. The method of claim 64, wherein the bias is applied by depressing said compression pump against the plunger.

66. The method of claim 65, wherein the compression pump comprises a spring, and said spring is operated to compress it.

67. A method of mixing a drug in a vial with a liquid from a reservoir to form a drug compound, and ejecting the drug compound from a reservoir in an drug delivery device, said drug delivery device having a needle, a plunger located within the reservoir, and a control valve therebetween, the method comprising:
(a) adjusting said control valve to establish fluid communication between the liquid in said reservoir and an interior of said drug vial via said control valve, the interior of said drug vial containing the drug;
(b) transferring the liquid from said reservoir to the interior of said drug vial, the liquid mixing with the drug to form the drug compound;
(c) transferring the drug compound to said reservoir;
(d) adjusting said control valve to terminate fluid communication between said reservoir and the interior of said drug vial;
(e) biasing said plunger against the drug compound in said reservoir to place the drug compound under pressure; and
(f) actuating a movement of said needle, the movement of said needle establishing fluid communication between the interior of said needle and the drug compound, the communication releasing the pressure of the drug compound in said reservoir and enabling said plunger to push the drug compound through said needle.
